(19) United States
(12) Patent Application Publication
(10) Pub. No.: US 2024/0026432 A1
(43) Pub. Date: Jan. 25, 2024

(54) POLYVINYL SULFONATE DETECTION AND REMOVAL FROM BIOMOLECULE COMPOSITIONS

(71) Applicant: AMGEN INC., Thousand Oaks, CA (US)

(72) Inventors: Neil Soice, Smi Valley, CA (US); Scott Kuhns, Newbury Park, CA (US); Andrew Csordas, Goleta, CA (US)

(21) Appl. No.: 18/029,995

(22) PCT Filed: Oct. 15, 2021
(86) PCT No.: PCT/US2021/055117

§ 371 Date: Apr. 3, 2023

Related U.S. Application Data
(60) Provisional application No. 63/093,120, filed on Oct. 16, 2020.

(51) Int. Cl. C12Q 1/6848; C12Q 1/6806

(52) U.S. Cl. CPC C12Q 1/6848; C12Q 1/6806

(57) ABSTRACT

The disclosure provides materials and methods for assessing the presence and level of polyanionic compounds in a sample that inhibit PCR reactions, including assays for the presence, and optionally the amount, of polyvinyl sulfonate, as well as materials and methods for the reduction or removal of such compounds from buffer solutions and from protein solutions using a variety of approaches, including titration-based techniques.

### CROSS-REFERENCE TO RELATED APPLICATIONS

This application claims priority to U.S. Provisional Patent Application No. 63/093,120, filed Oct. 16, 2020, which is hereby incorporated by reference in its entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55057A_Seqlisting.txt”, which was created on Oct. 4, 2021 and is 1,070 bytes in size. The subject matter of the Sequence Listing is incorporated herein in its entirety by reference.

### FIELD

The disclosure relates generally to the field of biomolecule purification and, more specifically, to protein purification.

### BACKGROUND

The potential of biologics and biosimilars is now being realized, with these therapeutic classes making ever-increasing contributions to the arsenal of treatments available for human and other animal diseases and conditions. These products, which include recombinant proteins, antibodies of various forms and fragments thereof that retain binding capacity, and vaccines are all expressed in host cells ranging from bacterial, yeast, animal cells such as mammalian cells, and continuous cell lines. As found in cell culture, these products are mixed with various contaminating substances, including fragments of host cell DNA. Moreover, residual amounts of host cell DNA can survive rigorous purification processes, remaining as a deleterious impurity in preparations of purified proteins such as biologics. Residual host cell DNA contained in a formulation of the protein to be administered to an animal such as a human patient could elicit an undesirable immune response or increase the risk of cancer. As a consequence, regulatory bodies around the world have placed limits on the concentration of host cell DNA contained in a formulation for administration to a human. The World Health Organization (WHO) and the European Union (EU) allow the amounts for up to 10 ng/dose of residual host cell DNA, while the U.S. Food and Drug Administration allow no more than 100 pg/dose. Accurate, precise and sensitive methods for detecting and quantitating low levels of host cell DNA are needed to ensure that purified protein formulations intended for administration fall below these thresholds. Moreover, the cell cultures used for the efficient production of these proteins contain impurities beyond host cell DNA. Some of these impurities, such as small molecule compounds, can have direct deleterious effects on the biologics and biosimilars being produced by these cells (i.e., the target proteins), such as by inhibiting the transcription or translation of a target protein, inhibiting the activity of the expressed target protein, or by interfering with efforts to measure or monitor the target protein as it undergoes a purification process.

Some polyanionic compounds typically found in viable cells used in culture-based recombinant target protein expression can be found as impurities in cell cultures. Additionally, some of these polyanionic compounds found in buffers and cell culture media are known inhibitors of various enzymes. Polyvinyl Sulfonate (PVS) is a polyanionic compound that is a known inhibitor of several enzymes, including RNA enzymes and DNA polymerases. PVS is also known to be present in preparations of 2-(N-morpholino)-ethanesulfonic acid (MES), a common buffer in biotherapeutic processing and purification operations. PVS may also be (undesirably) present in other buffer systems, e.g., Goods buffers, which utilize vinyl sulfonate as a starting material.

Thus, a need persists in the art for methods of accurately quantitating host cell DNA contained in protein formulations intended for administration to humans or other animals. Moreover, a need continues to exist for methods of reducing or removing such impurities from protein formulations intended for such administration.

### SUMMARY

The disclosure provides methods of assaying for polyanionic PCR inhibitors such as polyvinyl sulfonate compounds. These compounds inhibit a variety of enzymes, including nucleic acid polymerases such as DNA polymerases. Even the engineered forms of DNA polymerases that now dominate in PCR amplification methods are inhibited by these compounds, and the disclosure herein presents methods for detecting, and quantifying, these inhibitory compounds. The disclosure further provides methods for removing such compounds from buffers (e.g., MES and Goods buffers) as well as from protein solutions. These methods provide a significant advance in the processing of biologics and proteins in general by providing methods for monitoring the presence of a major class of PCR inhibitors that confound efforts to monitor the purity of biologics produced in cell culture, where host DNA must be monitored to ensure purity of the protein of interest sufficient to be used as a therapeutic in humans and/or other animals.

In one aspect, the disclosure provides a method for quantification of a polyanionic PCR inhibitor in a sample comprising: a) preparing a dilution series of a sample comprising at least four members; b) spiking each member of the dilution series with a constant amount of a template DNA distinguishable from host cell DNA; c) performing a PCR assay on each member of the dilution series and on the constant amount of the template DNA in the absence of any sample; d) generating a polyanionic inhibitor standard curve; e) comparing the PCR assay results of the dilution series to the PCR assay results of the constant amount of the template DNA in the absence of any sample; and f) identifying the concentration of polyanionic PCR inhibitor in the sample. In some embodiments, the concentration of polyanionic PCR inhibitor in the sample is a range defined by the concentration of polyanionic PCR inhibitor in the least diluted member of the dilution series showing complete spike recovery and the most diluted member of the dilution series not showing complete spike recovery. In some embodiments, the number of members in the dilution series is 5, 6, 7, 8, 9, 10, 12, 15, or 20, thereby narrowing the range of concentration of the polyanionic PCR inhibitor in the sample relative to the range provided by assaying fewer members of a dilution series. In some embodiments, the constant amount of a template DNA is at least 100 pg. In some embodiments, the polyanionic PCR inhibitor is a sulfone (sulfonate) compound, such as polyvinyl sulfonate. In some embodiments, polyvinyl sulfonate is the polyanionic PCR inhibitor used in generating the polyanionic inhibitor standard curve and the concentration of polyanionic PCR inhibitor in the sample is in units of polyvinyl sulfonate concentration equivalents. Some embodiments further comprise spiking a sample diluted sufficiently to recover amplification with one or more amounts of a polyanionic PCR inhibitor such as polyvinyl sulfonate to demonstrate that with added inhibitor, the recovery of amplification is impaired or lost.

Another aspect of the disclosure is drawn to a method for removing a polyanionic PCR inhibitor (a polyanionic impurity) from a buffer solution comprising: a) preparing a buffer solution of an acidic buffering species, a basic buffering species, or a combination thereof; b) contacting the buffer solution with an anion exchange medium; and c) separating the buffer solution from the polyanionic impurity, thereby removing the polyanionic impurity from the buffer solution. A related aspect of the disclosure provides a method for removing a polyanionic PCR inhibitor from a buffer solution comprising: a) preparing a buffer solution of an acidic buffering species, a basic buffering species, or a combination thereof; b) contacting the buffer solution with a mixed mode resin; and c) separating the buffer solution from the polyanionic impurity, thereby removing the polyanionic impurity from the buffer solution. In either of the foregoing methods for removing a polyanionic PCR inhibitor, the volume of buffer subjected to the method is not limiting and may extend from small analytical volumes in the range of milliliters to commercial scale buffer preparations involving many liters. In some embodiments of either removal method, the polyanionic impurity is a sulfone (sulfonate) compound, such as a polyvinyl sulfonate. In some embodiments of either removal method, the buffer solution is a Good's buffer solution, such as a 2-(N-morpholino)-ethanesulfonic acid (MES) buffer solution. In some embodiments of either removal method, the buffer solution comprises a buffering salt or acid species of the buffering salt. Some embodiments of each of the removal methods further comprise adding at least one modifying compound to the buffer solution. In some embodiments of either removal method, the modifying compound is a non-buffering salt, an excipient, or both.

In some embodiments of the removal method involving an anion exchange medium, the anion exchange medium is diethylaminoethyl-modified matrix, Dimethylaminoethyl-modified matrix, dimethylaminopropyl-modified matrix, polyethyleneimine-modified matrix, quaternized polyethyleneimine-modified matrix, fully quaternized amine-modified matrix, anion exchange modified diatomaceous earth-containing depth filters, anion exchange membrane adsorbers, salt tolerant anion exchange membrane adsorbers, Macro-Prep 25Q, TSK-Gel Q, Poros Q, Q Sepharose Fast Flow, Q HyperD, Q Zirconia, Source 30Q, Fractogel EMD TMAE, Express-Ion Q, DEAE Sepharose Fast Flow, Poros 50 D, Fractogel EMD DEAE (M), MacroPrep DEAE Support, DEAE Ceramic HyperD 20, Toyopearl DEAE 650 M, Capto Q, Sartobind Q membrane absorber, Posidyne charged membrane, Amberlite® (polyamine)-modified matrix, anion exchange-modified diatomaceous earth containing depth filters, anion exchange membrane adsorbers, salt tolerant anion exchange membrane adsorbers, Macro-Prep 25Q, TSK-Gel Q, Poros Q, Q Sepharose Fast Flow, Q HyperD, Q Zirconia, Source 30Q, Fractogel EMD TMAE, Express-Ion Q, DEAE Sepharose Fast Flow, Poros 50 D, Fractogel EMD DEAE (M), MacroPrep DEAE Support, DEAE Ceramic HyperD 20, Toyopearl DEAE 650 M, Capto Q, Sartobind Q membrane absorber, Posidyne charged membrane, Amberlite® (iminodiacetic acid)-modified matrix, Amberlite® Type I (trialkylbenzyl ammonium)-modified matrix, Amberlite® Type II (dimethyl-2-hydroxyethylbenzyl ammonium)-modified matrix, Dowex® (polyamine)-modified matrix, Dowex® Type I (trimethylbenzyl ammonium)-modified matrix, Dowex® Type II (dimethyl-2-hydroxyethylbenzyl ammonium)-modified matrix, Dowex® (mixed bed), Capto™ Adhere anion exchange multimodal medium, Capto® Adhere Anion Exchange Multi Mode, PPA Hypercel, HEA Hypercel, or Duolite® (polyamine)-modified matrix. In some embodiments of the removal method involving mixed mode resin, the mixed mode resin is Capto® Adhere Anion Exchange Multi Mode resin, PPA Hypercel resin, or HEA Hypercel resin. Any matrix known in the art is suitable for use in the removal methods, including but not limited to, cellulose, agarose, Sepharose®, methacrylic polymer, ceramic scaffolds with polymerized hydrogels, and proprietary matrices.

Some embodiments of the removal method involving an anion exchange medium provide an anion exchange medium that binds up to 15 mg, 9 mg, or 3 mg PVS per mL of anion exchange medium. Some embodiments of the removal method involving a mixed mode resin provide a mixed mode resin that binds up to 15 mg, 9 mg, or 3 mg PVS per mL of mixed mode resin. In some embodiments of the removal method involving an anion exchange medium, the anion exchange medium is a polycationic compound is a titrant that forms a complex with the polyanionic impurity (analyte). In some embodiments, the anion exchange medium is a quaternary ammonium-based polymer. In some embodiments of the removal method involving an anion exchange medium, the polycationic compound is added in an amount sufficient to at least reach the equivalence point in titrating the polyanionic impurity analyte. As used herein, the equivalence point is the point in a titration when sufficient titrant is added to bind all of the analyte and is a synonym for titration point.

Yet another aspect of the disclosure is drawn to a method for removing a polyanionic buffer impurity from a protein solution comprising: a) adjusting the pH of a protein solution containing an anionic buffer impurity to a pH less than the isoelectric point of the protein by no more than 4 pH units; b) contacting the protein solution with an anion exchange medium; and c) separating the protein from the anionic buffer impurity. In a related aspect, the disclosure provides a method for removing a polyanionic buffer impurity from a protein solution comprising: a) adjusting the pH of a protein solution containing an anionic buffer impurity to a pH less than the isoelectric point of the protein by no more than 4 pH units; b) contacting the protein solution with a mixed mode resin; and c) separating the protein from the anionic buffer impurity. In some embodiments of either of these pH-based removal methods, the pH is adjusted to be lower than the isoelectric point of the protein by no more than 2 pH units. In some embodiments of the pH-based removal method involving an anion exchange medium, the anion exchange medium is diethylaminoethyl-modified matrix, Dimethylaminoethyl-modified matrix, dimethylaminopropyl-modified matrix, polyethyleneimine-modified matrix, quaternized polyethyleneimine-modified matrix, fully quaternized amine-modified matrix, an anion exchange-modified diatomaceous earth-containing depth filter, an anion exchange membrane adsorber, a salt-tolerant anion exchange membrane adsorber, Macro-Prep 25Q, TSK-Gel Q, Poros Q, Q Sepharose Fast Flow, Q HyperD, Q Zirconia, Source 30Q, Fractogel EMD TMAE, Express-Ion Q, DEAE Sepharose Fast Flow, Poros 50 D, Fractogel EMD DEAE (M), MacroPrep DEAE Support, DEAE Ceramic HyperD 20, Toyopearl DEAE 650 M, Capto Q, Sartobind Q membrane absorber, Posidyne charged membrane, Amberlite® (polyamine)-modified matrix, Amberlite® (iminodiacetic acid)-modified matrix, Amberlite® Type I (trialkylbenzyl ammonium)-modified matrix, Amberlite® Type II (dimethyl-2-hydroxyethylbenzyl ammonium)-modified matrix, Dowex® (polyamine)-modified matrix, Dowex® Type I (trimethylbenzyl ammonium)-modified matrix, Dowex® Type II (dimethyl-2-hydroxyethylbenzyl ammonium)-modified matrix, Dowex® (mixed bed), Capto® Adhere Anion Exchange Multi Mode, PRA Hypercel, HEA Hypercel, or Duolite® (polyamine)-modified matrix. As noted above, any matrix known in the art is suitable for use in the methods according to the disclosure. In some embodiments of the pH-based removal method involving mixed mode resin, the mixed mode resin is Capto® Adhere Anion Exchange Multi Mode, PPA Hypercel, or HEA Hypercel.

Still another aspect of the disclosure is a titration method for detecting a polyanionic enzyme inhibitor in a buffer solution comprising: (a) contacting a buffer solution with a polycationic compound; (b) adding a polyanionic compound to the solution in (a), wherein the polyanionic compound exhibits a change in a detectable property when complexed to a polycationic compound compared to the uncomplexed polyanionic compound; (c) repeating steps (a) and (b) with varying concentrations of the buffer solution or varying concentrations of the polycationic compound; and (d) detecting the change in the detectable property at the titration point, thereby detecting the polyanionic enzyme inhibitor. In some embodiments, the buffer concentration is varied, thereby creating a dilution series of the buffer. In some embodiments, the concentration of the polycationic compound is varied. In some embodiments, the polyanionic enzyme inhibitor is polyvinyl sulfonate or a derivative thereof. In some embodiments, the polycationic compound is a pH-independent polycationic compound or a pH-dependent polycationic compound. In some embodiments, the pH-independent polycationic compound is a quaternary ammonium-based polymer. In some embodiments, the pH-dependent polycationic compound is a polyamine. In some embodiments, the quaternary ammonium-based polymer is hexadimethrine bromide (HDBr), poly(diallyl)dimethylammonium chloride (pDADMAC), or methylglycol chitosan. In some embodiments, the quaternary ammonium-based polymer is hexadimethrine bromide (HDBr) or poly(diallyl)dimethylammonium chloride (pDADMAC). In some embodiments, the quaternary ammonium-based polymer is hexadimethrine bromide (HDBr).

In some embodiments, the polyanionic compound is a dye. In some embodiments, the dye is Eriochrome Black T (ECBT), Eriochrome Blue Black R (Calcon) or Sulfonazo sodium salt. In some embodiments, the dye is Eriochrome Black T (ECBT). In some embodiments, the buffer is a Good's buffer. In some embodiments, the Good's buffer comprises a polyethane sulfonic acid derivative or a polypropane sulfonic acid derivative. In some embodiments, the Good's buffer is MES, Bis-tris methane, ADA, Bis-tris propane, PIPES, ACES, MOPSO, Cholamine chloride, MOPS, BES, AMPB, HEPES, DIPSO, MOBS, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, CAPSO, AMP, CAPS, or CABS. In some embodiments, the uncomplexed polyanionic compound is detected using fluorometry or spectrophotometry. In some embodiments, the method further comprises determining the concentration of the polyanionic enzyme inhibitor from the quantity of polyanionic compound required to detect the change in the detectable property.

Another aspect of the disclosure is drawn to a method for quantification of a polyanionic PCR inhibitor in a sample comprising: (a) contacting a sample comprising a polyanionic PCR inhibitor with at least one aliquot of a polycationic compound; (b) adding a polyanionic indicator dye in an amount sufficient to detect the free form of the dye; and (c) quantifying the polyanionic PCR inhibitor based on the amount of polycationic compound needed to detect the free form of the polyanionic indicator dye. A related aspect of the disclosure is directed to a method of removing a polyanionic impurity in a sample comprising: (a) contacting a fluid comprising a polyanionic impurity with a polycationic counterion; and (b) separating the fluid from the polyanionic impurity complexed to the polycationic counterion, thereby removing the polyanionic impurity from the fluid. In some embodiments, the polyanionic impurity is a polyanionic PCR inhibitor. In some embodiments, the complex of polyanionic impurity and polycationic counterion is removed by precipitation. In some embodiments, the polycationic counterion is derivatized by attachment to a member of a binding pair or a magnetic particle to facilitate removal of the complex of polyanionic impurity and polycationic counterion from the fluid. Exemplary binding pairs include, but are not limited to, antigen/antibody pairs, biotin/streptavidin, magnetic particle/iron-containing material, polyhistidine/metal ion (e.g., nickel) pairs, and the like.

Other features and advantages of the disclosed subject matter will be apparent from the following detailed description and figures, and from the claims.

### BRIEF DESCRIPTION OF THE DRAWINGS

FIG. 1. DNA assay spike recovery inhibition versus PVS standards.

FIG. 2. Dilution series spike recovery analysis compared to standard curve to provide PVS concentrations.

FIG. 3. Spike recovery data for MES fractions.

FIG. 4. Spike recovery data for MES fractions.

FIG. 5. Spike recovery data for MES fractions using AEX resin.

FIG. 6. Assay results establishing that Polyvinyl Sulfonate (PVS) is a potent inhibitor of the qPCR-mediated DNA assay.

FIG. 7. Chromatography method for PVS spike challenge.

FIG. 8. (a) Chemical structure of 2-(N-morpholino)-ethane sulfonic acid (i.e., MES) shown as the acidic form, MES hydrate, and as the basic form, MES sodium salt. (b) Chemical reactions leading to compounds capable of inhibiting enzymes active on nucleic acids, such as RNA enzymes. FIG. 1(b) is adapted from a figure in Smith, et al. Journal of Biological Chemistry 2003, 20934-20938.

FIG. 9. (a) Varying the concentration of polyvinyl sulfonate (PVS) between 0-1.0 ppm revealed a linear calibration curve for two different lots of PVS standards obtained from Sigma-Aldrich, which were provided as 30 wt % aqueous solutions. The concentration of PVS was found to vary significantly lot-to-lot. It is contemplated, however, that the concentration of a particular lot can be adjusted by dilution to serve as a suitable standard. (b) Titration curves using hexadimethrine bromide (HDBr) to titrate PVS were constructed across the range of PVS concentrations of 0-1.0 ppm. A linear range of about 1.5 orders of magnitude was found.

FIG. 10. (a) Schematic for quantitation by titration of PVS with HDBr with spectroscopic endpoint detection. The reaction scheme depicts complexation between PVS and HDBr driven by attractive electrostatic interactions. At the endpoint of the titration, an indicator compound (nD⁻) undergoes a change in absorbance properties upon association with neighboring HDBr charge sites. The blue circular “plus” and “minus” symbols in FIG. 3(a) refer to background salts in solution. (b) The plot shows the evolution of the solution absorbance for a blank sample (i.e., 50 mM borate buffer, pH 8.5, supplemented with EBT indicator compound), measured on a benchtop absorbance spectrometer, as HDBr is incrementally titrated into solution. With increasing HDBr concentration, the indicator absorbance at 665 nm decreases with a concomitant shift in the absorbance maximum from about 630 to 593 nm.

FIG. 11. Titration curves plotting the normalized, volume-corrected absorbance at 665 nm for a series of PVS standard solutions.

FIG. 12. (a) Plot of the volume-corrected solution absorbance at 665 nm against the HDBr (titrant) mass for three different PVS standards prepared in MES matrix blank. (b) A comparison of the titration curve inflection points between PVS standards prepared in 50 mM sodium borate (green triangles) and MES mixed with 50 mM sodium borate (black squares).

FIG. 13. A comparison of the titration curve inflection points for PVS standards prepared in MES matrix blank (black squares), a negative control lot of MES (SLBT8755; blue diamond), and the MES lot that caused initial qPCR invalid assays (Lot #I; red circle).

FIG. 14. Representative profile for titration of a blank standard (100 mM carbonate buffer supplemented with 1.25 μg/mL Eriochrome Black T (EBT) indicator dye) with 0.04 mg/mL hexadimethrine bromide (HDBr; black trace) and the corresponding first derivative (red trace).

FIG. 15. (a) Plot of titration endpoint volume versus the concentration of PVS spiked into 50 mM MES dissolved in 100 mM carbonate buffer. (b) Plot of titration endpoint volume versus the concentration of PVS for standard samples prepared in 100 mM carbonate buffer.

FIG. 16. Representative titration curves for (A,B) PVS standard solutions prepared at 0 (A) or 0.75 (B) μg/mL in 100 mM carbonate buffer; and (C,D) PVS spiked at 0 (C) or 0.70 (D) μg/mL into 50 mM MES (sample H in Table 10) prepared in 100 mM carbonate buffer.

### DETAILED DESCRIPTION

MES (2-(N-morpholino)-ethanesulfonic acid) buffer and other Good's buffers are common buffers in biologics processes, enabling the control of pH around pH 6 (pKa of MES is 6.15). The synthesis of MES involves the Michael Addition of a morpholine ring to vinyl sulfonate. A common side reaction is the oligomerization/polymerization of vinyl sulfonate, forming the polyanionic polyvinyl sulfonate. Polyvinyl sulfonate is a potent inhibitor of the quantitative polymerase chain reaction (qPCR) assay used to quantify residual host cell deoxyribonucleic acid (DNA), resulting in failed spike recovery in assay controls and invalid test results. The level of PVS that inhibits the DNA assay is far below the level of PVS that would raise safety concerns. The inability to determine the host cell DNA content with a valid spike recovery control, however, impacts lot characterization for purified proteins such as biologics, as well as the release and disposition of such lots.

The disclosure provides methods of assaying for polyanionic compounds present in cell culture media or fluids derived therefrom, such as mammalian cell culture media or fluids, that are sensitive to low levels of the polyanionic compounds. The disclosure reveals that the low levels of polyanions in cell culture fluids confound efforts to monitor the purification of proteins in general and biologics in particular, increasing the time and expense required to gain approval for therapeutic use. The methods of the disclosure provide simple and efficient approaches for monitoring the reduction of polyanionic impurities to vanishingly small, or non-existent, concentrations. The recent explosion of commercial production processes for proteins such as biologics, i.e., therapeutic antibodies and antibody fragments, has placed increasing pressure on the industry to develop protein purification processes that efficiently result in high yields of pure protein product suitable for formulation in therapeutics, and the methods of the disclosure provide an answer in facilitating the simple and effective monitoring of polyanionic impurities often seen in protein-containing cell culture fluids of varying purities. The knowledge disclosed herein that polyanionic impurities are present in protein-containing fluids, e.g., solutions, that have been conventionally regarded as purified has led to the disclosed methods of reducing or removing the polyanionic impurities from the protein-containing fluids, e.g., solutions. The disclosure reveals that, even in such fluids, polyanionic impurities can be found at levels that interfere with enzymes, e.g., DNA polymerases, often used to assay the purity of a protein solution. For example, qPCR is frequently used to monitor levels of host cell DNA in a purification process designed to obtain protein from cell culture, e.g., mammalian cell culture. In view of the discovery of the presence of even trace quantities of polyanionic impurities present during the purification process that interfere with efforts to monitor purity, the disclosure provides methods of reducing or removing polyanionic impurities from protein solutions regarded as pure in the state of the art.

Polyanionic compounds such as poly(vinylsulfonic acid) (PVS) are polymeric impurities in Good's buffers such as MES buffer. These polyanionic compounds, e.g., PVS, are present in such buffers at low levels in the range of parts per million relative to the buffering compound such as MES. The presence of these impurities in Good's buffers is a significant concern because such buffers are used in the manufacture of therapeutic proteins, and these impurities, and in particular PVS, are potent polymerase inhibitors that can interfere with quantitative PCR (qPCR) detection of DNA. Measures of host cell nucleic acids (e.g., DNA) in formulations of therapeutic proteins purified from culture are routinely required to assess the safety of therapeutics intended for administration to humans. Thus, the presence of polyanionic compounds such as PVS in Good's buffers such as MES can cause batches of therapeutic protein to fail acceptance criteria for human administration by interfering with qPCR detection of host cell DNA.

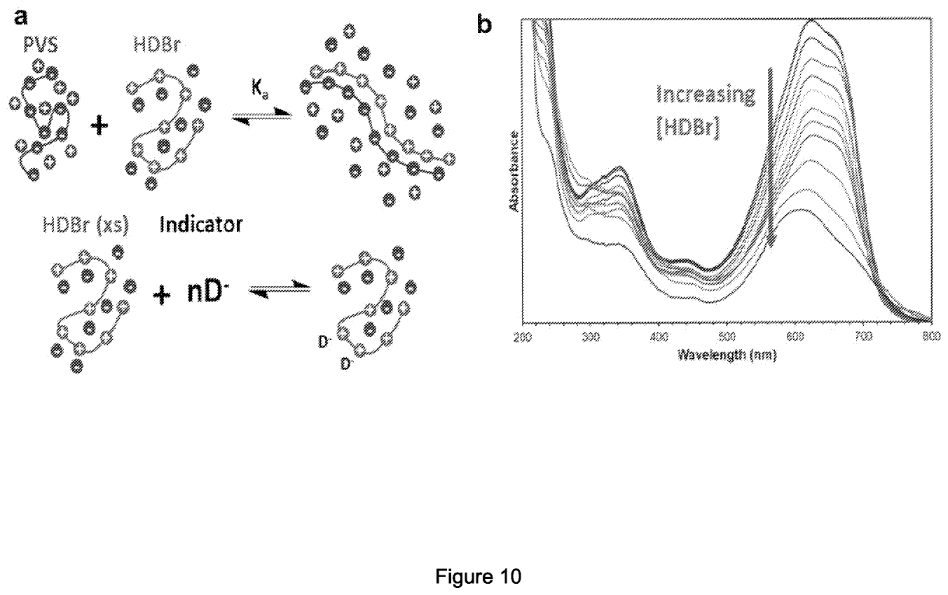

Disclosed herein are methods involving titration based on complexation of the analyte (e.g., PVS) with an oppositely charged, high molecular weight titrant. This interaction results in an exceedingly high equilibrium association constant (Ka) and the endpoint can be detected spectroscopically (e.g., colorimetrically or photometrically). A summary of the detection scheme, applied to the titration of PVS with hexadimethrine bromide (HDBr), an exemplary titrant, is provided in FIG. 10.

The methods of the disclosure include methods of confirming the accuracy of nucleic acid enzyme-based assays of host cell DNA as an impurity in protein formulations. For example, the methods disclosed herein are useful in confirming nucleic acid enzyme-based assays of host cell DNA, such as polymerase chain reaction (i.e., PCR). An exemplary PCR assay useful in the disclosed methods is quantitative PCR or qPCR, which provides a rapid, inexpensive, accurate, precise and sensitive method for determining the quantity of DNA in a sample. Accordingly, preferred methods of confirming the concentration of host cell DNA impurity in a protein fluid, solution, preparation or formulation involves the quantification of DNA in a sample, such as a cell culture sample, using qPCR and comparison to a polyanionic PCR inhibitor standard curve to determine the concentration of a polyanionic PCR inhibitor in the protein fluid, solution, preparation or formulation to confirm host cell DNA assay results. Related aspects of the disclosure address the problem of nucleic acid enzyme inhibition by providing methods of reducing or removing such inhibitors from cell culture fluids of varying purity, i.e., protein-containing fluids or solutions, and by removing such inhibitors from buffers in which such proteins may be placed.

The methods disclosed herein are useful in reducing or removing one or more polyanionic compounds, such as polyanionic compounds found in cell culture, e.g., mammalian cell culture, or in buffers found in therapeutic formulations such as in 2-(N-morpholino)-ethanesulfonic acid (MES) or Goods buffers. An exemplary group of polyanionic compounds reduced or removed according to methods of the disclosure are sulfonate compounds, as typified by polyvinyl sulfonate (i.e., polyethylene sulfonate). The disclosure contemplates the reduction or removal of polyanionic compounds regardless of size or range of sizes of the relevant polymer or polymers. Polyanionic impurities that can be removed using the methods of the disclosure also include polyoxometalates (i.e., POMs), proteoglycans (storage depots), glycosaminoglycans (e.g., heparin, chondroitin sulfates, dextran sulfate), polyglutamate, polysaccharides, actin microfilaments and actin microtubules, polyvinyl sulfonates, polyacrylic acid, and inositol phosphates.

The methods according to the disclosure use anion exchange media to separate polyanionic impurities from a protein, e.g., a biologic, being purified. Any anion exchange medium known in the art may be used in the disclosed methods, including, but not limited to, weakly basic groups such as diethylaminoethyl (DEAE) and dimethylaminoethyl (DMAE), dimethylaminopropyl (DMAP), or strongly basic groups such as quaternary aminoethyl (Q), trimethylammoniumethyl (TMAE), and quaternary aminoethyl (QAE)) can be used in anion exchange. Exemplary anion exchange media are GE Healthcare Q-Sepharose FF®, Q-Sepharose BB®, Q-Sepharose XL®, Q-Sepharose HP®, Mini Q™, Mono Q, Mono P DEAE Sepharose FF®, Source™ 15Q, Source™ 30Q, Capto Q™, Streamline DEAE®, Streamline QXL®; Applied Biosystems Poros™ HQ 10 and 20 μm self-pack, Poros™ HQ 20 and 50 μm, Poros™ PI 20 and 50 μm, Poros™ D 50 μm Tosohaas Toyopearl® DEAE 650S M and C, Super Q 650, QAE 550C; Pall Corporation DEAE HyperD™, Q Ceramic HyperD™, Mustang Q membrane absorber: Merck KG2A Fractogel DMAE®, FractoPrep DEAE, FractoPrep TMAE, Fractogel EMD DEAE®, Fractogel EMD TMAE®; and Sartorious Sartobind Q® membrane absorber. Any mixed mode or multimodal medium known in the art that comprises an anion exchanger, may be used in the disclosed methods, including, but not limited to, Capto® Adhere Anion Exchange Multi Mode, PRA Hypercel, or HEA Hypercel, media. In addition, the disclosed methods may include the use of polyanion-binding proteins such as α-synuclein, tRNA/rRNA methyltransferase, and/or small heat shock proteins. In some preferred embodiments, Hybrid Purifier® is used as an anion exchange medium in addition to functioning as a depth filter. Also preferred is a Viresolve pre-filter (VPF) for use as an anion exchange medium.

Methods of the disclosure useful in confirming the accuracy of host cell DNA assays of, e.g., cell culture samples may use any enzyme-based nucleic acid assay, such as any of the variant forms of PCR. A preferred type of PCR for use in such methods is qPCR. PCR, including qPCR, is well-suited to the detection and quantification of DNA from cultured cells, such as the host cell DNA found as an impurity in tissue culture fluids. An advantage of qPCR is the capacity to detect and quantitate an increase in fluorescence occurring after each round of PCR. To provide this capacity, forward and reverse primers are designed to flank a target DNA sequence of interest, and a target specific probe is designed to hybridize to a complementary sequence between the two primers. The probe consists of an oligonucleotide sequence with a fluorophore molecule at its 5′ end and a quencher molecule at its 3′ end. When the fluorophore is in close proximity to the quencher, fluorescence is minimized. In the presence of the target sequence, however, the probe can anneal to the target sequence and subsequently become cleaved by the exonuclease activity of the Taq polymerase. Once the probe is cleaved as a result of extension of the forward primer, the probe's fluorophore is no longer quenched, and this results in an increase in fluorescence as a direct result of the presence of the target DNA sequence. Fluorescence is monitored during each cycle of qPCR during the extension phase of thermal cycling and threshold cycles are determined for each reaction. The threshold cycle is the cycle at which the fluorescence from a given reaction is significantly above the background fluorescence. Threshold cycle values are inversely proportional to amount of starting DNA in a reaction. The threshold cycle value of each sample is compared to those from a standard curve, allowing quantification of samples with unknown quantities of DNA.

Any set of primers functional in qPCR, as can readily be determined by those of skill in the art, is suitable for use in the methods of the disclosure. Exemplary qPCR primers are primers derived from, and thereby specifically hybridizing to, a repetitive sequence specific to CHO cells. The CHO-cell specific sequence targeted is a 68-base region as follows: 5′-GAAATCGGGCTGCCTGAGTCCCGAGTGCGGGTGTGGTTTCAGAACCGCCGAAGTCGTTC GGGGATGGT-3′ (SEQ ID NO: 1). The 5′ end of this sequence has the same sequence as the forward primer, the 3′ end of the sequence is the complement of the reverse primer, and the fluorophore labeled probe targets a region between these sequences. The forward, reverse, and probe sequences are as follows: RepA forward primer: 5′-GAA ATC GGG CTG CCT GAG T-3′ (SEQ ID NO: 2); RepA reverse primer: 5′-ACC ATC CCC GAA CGA CTT C-3′ (SEQ ID NO: 3); and RepA probe: 5′-CC GAG TGC GGG TGT GGT TT-3′ (SEQ ID NO: 4). The RepA probe contains a fluorophore group at the 5′-end and a quencher group at the 3′-end.

The qPCR assays for host cell DNA impurities were conducted in accordance with conventional procedures. Following DNA extraction from samples, qPCR reagents including qPCR primers, a DNA polymerase, such as a thermostable polymerase (e.g., Taq® DNA polymerase) and appropriate quantities of the required nucleoside triphosphates, as would be known in the art, were added. To some samples, a DNA spike control was added in the form of a DNA that is amenable to qPCR amplification. The spike amount added to the spiked samples was 100 pg of CHO genomic DNA. Other samples remained unspiked. The difference in results between a spiked sample and an unspiked sample allowed for the calculation of the percent spike recovery. In other words, the percent spike recovery is given by [(spiked result in pg−unspiked result in pg)/spike amount in pg]×100.

Fluorescence can be measured from individual wells of a 96-well plate. Because this measurement is obtained prior to reaction completion at the end of 40 thermocycles, it is possible to determine the degree of PCR that has occurred in real time. PCR is measured by monitoring the fluorescence increase as a function of cycle number.

qPCR can be carried out on instruments such as the QuantStudio 7 real-time qPCR instrument. Fluorescence can be monitored as a function of cycle number with fluorescence emission signal detection occurring the during the extension phase of amplification. A normalized reporter signal (Rn) is generated at each cycle for each sample run on the plate. The threshold cycle values from each well are compared to a standard curve (linear regression of threshold cycle versus log(input mass of DNA in each reaction) to allow for interpolation of unknown values.

The use of qPCR to assay nucleic acids has become widespread and, as a result, there are now kits available to facilitate such assays. Any known protocol and any kit known in the art may be used in the methods of the disclosure. An exemplary protocol is the protocol for TaqMan® qPCR method for residual host cell DNA quantitation described Example 1 and in Verardo et al., Biotechnol. Prog. 28:428-434 (2012), incorporated in relevant part by reference herein. An exemplary kit is the PrepSEQ® Residual DNA Sample Preparation Kit (Applied Biosystems®, Beverly, MA).

The methods of the disclosure useful in confirming the presence and amount of host cell DNA impurities in protein-containing fluids were developed to address the problem, disclosed herein, of relatively low levels of polyanionic inhibitors of enzyme-based nucleic acid assays persisting in protein-containing fluids in purification processes. Some embodiments of these methods achieve remarkable sensitivities while retaining the capacity to deliver accurate and precise results by serially diluting the sample and by comparison of results to standard curves. A sample, such as a sample from a cell culture, is serially diluted according to any scheme known in the art, provided that the degree of dilution of each aliquot of the sample is known. A suitable dilution scheme is a constant two-fold dilution in which an aliquot of a sample is diluted with an equal volume of a suitable solution, such as a PCR buffer solution, to create a 2:1 dilution. An aliquot of this dilution is then itself diluted 2:1, resulting in a series of dilutions ranging from 2:1 to 2ⁿ:1, where n is the number of aliquots. Determining the actual number of aliquots of diluted sample is within the skill in the art; typically the number of aliquots will range from 4-10 aliquots. The methods of the disclosure further contemplate adding, or spiking, a control template DNA to monitor amplification levels in the samples and dilutions thereof. The control template DNA or spike control is distinguishable from the host cell DNA impurity that may be present in a sample or dilution thereof, and the spike control will have PCR primer binding sites. The control template DNA or spike controls may be added to the original dilution series of the sample, to a separated portion of each aliquot of the original dilution series, or to a second dilution series of the sample prepared in conjunction with the original dilution series.

The use of dilution series in the methods of the disclosure designed to confirm whether host cell DNA impurities are present may appear to be counterintuitive or counterproductive at first, because in diluting the sample, one is diluting any impurity in that sample as well, presumably making it harder to detect and quantify. The tremendous sensitivity of enzyme-based nucleic acid assays such as PCR (e.g., qPCR) are capable of overcoming such dilutions and detecting vanishingly small amounts of host cell DNA impurities, however. Moreover, there is another reason for including dilution series in the methods of the disclosure. The serial dilution of a sample also serially dilutes any inhibitors of the enzymes used in these sensitive enzyme-based nucleic acid assays, such as DNA polymerases. As noted herein, the methods disclosed herein are based, in part, on the discovery of relatively low levels of polyanionic inhibitors of the enzymes used in nucleic acid assays, termed polyanionic PCR inhibitors for convenient reference. In preparing a dilution series of a sample, a dilution series of any polyanionic PCR inhibitors is also necessarily prepared. This provides an opportunity to determine the level of sample dilution at which there is a release of inhibition and a resumption of enzyme-based amplification due to DNA polymerase-mediated polymerization. Because there are a finite number of dilutions in the series, the results may yield a range for the concentration of a polyanionic PCR inhibitor, going from the least diluted sample demonstrating recovery of PCR activity, or spike recovery, to the most diluted sample that still exhibits inhibition of PCR activity. Those of skill in the art are equipped to narrow or expand the concentration range of the detected and quantified inhibitor by adding or subtracting aliquots from the dilution series. The skilled person also understands that a standard curve of a polyanionic PCR inhibitor permits conversion of relative dilutions to actual concentrations, based on a standard curve constructed from serial dilutions of pure polyanionic PCR inhibitor subjected to enzyme-based nucleic acid assay of control template DNA (spike controls) in the presence of needed reagents (e.g., TaqMan® Universal PCR Master Mix, Applied Biosystems) but the absence of any sample or dilution thereof. The standard curve identifies an absolute concentration of polyanionic PCR inhibitor with an observed level of nucleic acid amplification, which can then be carried over to the results seen with the sample dilution series. The methods contemplate generation of a standard curve using any known polyanionic PCR inhibitor, with polyvinyl sulfonate (polyethylene sulfonate) being a preferred polyanionic PCR inhibitor suitable for use in constructing a standard curve. Where PVS is used, concentrations are expressed in terms of PVS equivalents. In many instances, the PVS equivalents are actual concentrations of PVS in a sample or its dilutions because the identity of the polyanionic PCR inhibitor is known to be PVS.

The samples subjected to the methods of the disclosure are cell culture fluids or are fluids derived from cell culture fluid during processes for purifying proteins such as biologics and biosimilars. A sample may be of any volume suitable for detecting impurities and may be obtained from an ongoing cell culture, from continuous effluent from a cell culture, or from a discharged batch of cell culture. The sample may be obtained and processed without delay or may be obtained from a holding tank or maintained in storage at a suitable temperature, typically 4° C.

In addition to the methods of confirming whether formulations of varying purity that contain proteins produced in cell culture have host DNA impurities, the disclosure provide methods for reducing or removing the impurity based, in part, on the discovery that partially purified protein formulations can have levels, albeit frequently low levels in highly purified protein formulations, of polyanionic PCR inhibitors that must be addressed to satisfy regulatory bodies responsible for ensuring the quality of pharmaceutical formulations. Thus, another aspect of the disclosure is drawn to methods of removing a polyanionic PCR inhibitor such as PVS from a protein-containing solution. Informed by the disclosure of the existence of relatively low levels of polyanionic PCR inhibitors in protein-containing solutions obtained from cell cultures and purification processes for those proteins, the skilled worker would be able to contact the sample (or dilution thereof) with any known anion exchange medium to bind the polyanionic PCR inhibitor, leading to its separation and removal from the protein-containing sample or dilution thereof. To facilitate the reduction or removal effort, the pH of samples or dilutions thereof are adjusted to be 2-4 pH units below the pl of the protein target in the sample or the protein being purified. In this pH range, the protein of interest will not have a net negative charge, but PVS will exhibit its full negative charge, resulting in PVS, but not the protein of interest, readily binding to an anion exchange resin known in the art.

The protein being purified, such as a recombinant protein or polypeptide, can be homopolymeric or heteropolymeric, and can be of scientific or commercial interest, including protein-based therapeutics. Biomolecules (e.g., proteins such as biologics or biosimilars) of interest include, among other things, secreted proteins, non-secreted proteins, intracellular proteins or membrane-bound proteins. Biomolecules of interest can be produced by recombinant animal cell lines using cell culture methods and may be referred to as “recombinant proteins”. The expressed protein(s) may be produced intracellularly or secreted into the culture medium from which it can be recovered and/or collected. The term “isolated protein” or “isolated recombinant protein” refers to a polypeptide or protein of interest, that is purified away from proteins or polypeptides or other impurities that would interfere with its therapeutic, diagnostic, prophylactic, research or other use. Biomolecules of interest include proteins that exert a therapeutic effect by binding a target, particularly a target among those listed below, including targets derived therefrom, targets related thereto, and modifications thereof.

By “purifying” is meant increasing the degree of purity of the protein in the composition by removing (partially or completely) at least one product-related impurity from the composition. Recovery and purification of proteins is accomplished by any downstream process, particularly the harvest operation, resulting in a more “homogeneous” protein composition that meets yield and product quality targets (such as reduced product-related impurities and increased product quality).

As used herein, the term “isolated” means (i) free of at least some other proteins or polynucleotides with which it would normally be found, (ii) is essentially free of other proteins or polynucleotides from the same source, e.g., from the same species, (iii) separated from at least about 50 percent of polypeptides, polynucleotides, lipids, carbohydrates, or other materials with which it is associated in nature, (iv) operably associated (by covalent or noncovalent interaction) with a polypeptide or polynucleotide with which it is not associated in nature, or (v) does not occur in nature.

Biomolecules (e.g., proteins) of interest include “antigen-binding proteins”. Antigen-binding protein refers to proteins or polypeptides that comprise an antigen-binding region or antigen-binding portion that has affinity for another molecule to which it binds (antigen). Antigen-binding proteins encompass antibodies, peptibodies, antibody fragments, antibody derivatives, antibody analogs, fusion proteins (including single-chain variable fragments (scFvs) and double-chain (divalent) scFvs), muteins, multispecific proteins, and bispecific proteins.

An scFv is a single chain antibody fragment having the variable regions of the heavy and light chains of an antibody linked together. See U.S. Pat. Nos. 7,741,465, and 6,319,494 as well as Eshhar et al., Cancer Immunol Immunotherapy (1997) 45: 131-136. An scFv retains the parent antibody's ability to specifically interact with target antigen.

The term “antibody” includes reference to both glycosylated and non-glycosylated immunoglobulins of any isotype or subclass or to an antigen-binding region thereof that competes with the intact antibody for specific binding. Unless otherwise specified, antibodies include human, humanized, chimeric, multi-specific, monoclonal, polyclonal, heterolgG, bispecific, and oligomers or antigen binding fragments thereof. Antibodies include the IgG1-, IgG2- IgG3- or IgG4-type. Also included are proteins having an antigen binding fragment or region such as Fab, Fab′, F(ab′)2, Fv, diabodies, Fd, dAb, maxibodies, single chain antibody molecules, single domain V_{H}H, complementarity determining region (CDR) fragments, scFv, diabodies, triabodies, tetrabodies and polypeptides that contain at least a portion of an immunoglobulin that is sufficient to confer specific antigen binding to a target polypeptide.

Also included are human, humanized, and other antigen-binding proteins, such as human and humanized antibodies, that do not engender significantly deleterious immune responses when administered to a human.

Modified proteins are also included, such as are proteins modified chemically by a non-covalent bond, covalent bond, or both a covalent and non-covalent bond. Also included are proteins further comprising one or more post-translational modifications which may be made by cellular modification systems or modifications introduced ex vivo by enzymatic and/or chemical methods or introduced in other ways.

“Multispecific protein” and “multispecific antibody” are used herein to refer to proteins that are recombinantly engineered to simultaneously bind and neutralize at least two different antigens or at least two different epitopes on the same antigen. For example, multispecific proteins may be engineered to target immune effectors in combination with targeting cytotoxic agents to tumors or infectious agents. Multispecific proteins include bispecific antibodies, tetravalent bispecific antibodies, multispecific proteins without antibody components such as dia-, tria- or tetrabodies, minibodies, and single chain proteins capable of binding multiple targets. Coloma, M. J., et al., Nature Biotech. 15 (1997) 159-163.

The most common and most diverse group of multispecific proteins are those that bind two antigens, referred to herein as “bispecific”, “bispecific constructs”, “bispecific proteins”, and “bispecific antibodies”. Bispecific proteins can be grouped in two broad categories: immunoglobulin G (IgG)-like molecules and non-IgG-like molecules. IgG-like molecules retain Fc-mediated effector functions, such as antibody-dependent cell mediated cytotoxicity (ADCC), complement-dependent cytotoxicity (CDC), and antibody-dependent cellular phagocytosis (ADCP), the Fc region helps improve solubility and stability and facilitate some purification operations. Non-IgG-like molecules are smaller, enhancing tissue penetration (see Sedykh et al., Drug Design, Development and Therapy 18(12), 195-208, 2018; Fan et al., J Hematol & Oncology 8:130-143, 2015; Spiess et al., Mol Immunol 67, 95-106, 2015; Williams et al., Chapter 41 Process Design for Bispecific Antibodies in Biopharmaceutical Processing Development, Design and Implementation of Manufacturing Processes, Jagschies et al., eds., 2018, pages 837-855. Bispecific proteins are sometimes used as a framework for additional components having binding specificities to different antigens or numbers of epitopes, increasing the binding specificity of the molecule.

The formats for bispecific proteins, which include bispecific antibodies, are constantly evolving and include, but are not limited to, single chain antibodies, quadromas, knobs-in-holes, cross-MAbs, dual variable domains IgG (DVD-IgG), IgG-single chain Fv (scFv), scFv-CH3 KIH, dual action Fab (DAF), half-molecule exchange, Kλ-bodies, tandem scFv, scFv-Fc, diabodies, single chain diabodies (scDiabodies), scDiabodies-CH3, triple body, miniantibody, minibody, TriBi minibody, tandem diabodies, scDiabody-HAS, Tandem scFv-toxin, dual-affinity retargeting molecules (DARTs), nanobody, nanobody-HSA, dock and lock (DNL), strand exchange engineered domain SEEDbody, Triomab, leucine zipper (LUZ-Y), XmAb®; Fab-arm exchange, DutaMab, DT-IgG, charged pair, Fcab, orthogonal Fab, IgG(H)-scFv, scFV-(H)IgG, IgG(L)-scFV, IgG(L1H1)-Fv, IgG(H)-V, V(H)-IgG, IgG(L)-V V(L)-IgG, KIH IgG-scFab, 2scFV-IgG, IgG-2scFv, scFv4-Ig, Zybody, DVI-Ig4 (four-in-one), Fab-scFv, scFv-CH-CL-scFV, F(ab′)2-scFv2, scFv-KIH, Fab-scFv-Fc, tetravalent HCAb, scDiabody-Fc, diabody-Fc, intrabody, ImmTAC, HSABody, IgG-IgG, Cov-X-Body, scFv1-PEG-scFv2, bi-specific T cell engagers (BiTE®s) and half-life extended bispecific T cell engagers (HLE BiTE®s), heterolg BiTE®s (Fan supra; Spiess supra; Sedykh supra; Seimetz et al., Cancer Treat Rev 36(6) 458-67, 2010; Shulka and Norman, Chapter 26 Downstream Processing of Fc Fusion Proteins, Bispecific Antibodies, and Antibody-Drug Conjugates, in Process Scale Purification of Antibodies Second Edition, Uwe Gottschalk editor, p559-594, John Wiley & Sons, 2017; Moore et al., MAbs 3:6, 546-557, 2011). Biomolecules (e.g., proteins) of interest may also include recombinant fusion proteins comprising, for example, a multimerization domain, such as a leucine zipper, a coiled coil, an Fc portion of an immunoglobulin, and the like. Also included are proteins comprising all or part of the amino acid sequences of differentiation antigens (referred to as CD proteins) or their ligands or proteins substantially similar to either of these.

Biomolecules (e.g., proteins such as biologics and biosimilars) of interest also include genetically engineered receptors such as chimeric antigen receptors (CARs or CAR-Ts) and T cell receptors (TCRs), as well as other proteins comprising an antigen binding molecule that interacts with that targeted antigen. CARs can be engineered to bind to an antigen (such as a cell-surface antigen) by incorporating an antigen binding molecule that interacts with that targeted antigen. CARs typically incorporate an antigen binding domain (such as scFv) in tandem with one or more costimulatory (“signaling”) domains and one or more activating domains.

In some embodiments, biomolecules of interest may include colony stimulating factors, such as granulocyte colony-stimulating factor (G-CSF). Such G-CSF agents include, but are not limited to, Neupogen® (filgrastim) and Neulasta® (pegfilgrastim). Also included are erythropoiesis stimulating agents (ESA), such as Epogen® (epoetin alfa), Aranesp® (darbepoetin alfa), Dynepo® (epoetin delta), Mircera® (methoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin zeta, epoetin theta, and epoetin delta, epoetin omega, epoetin iota, tissue plasminogen activator, GLP-1 receptor agonists, as well as the molecules or variants or analogs thereof and biosimilars of any of the foregoing.

In some embodiments, biomolecules of interest may include proteins that bind specifically to one or more CD proteins, HER receptor family proteins, cell adhesion molecules, growth factors, nerve growth factors, fibroblast growth factors, transforming growth factors (TGF), insulin-like growth factors, osteoinductive factors, insulin and insulin-related proteins, coagulation and coagulation-related proteins, colony stimulating factors (CSFs), other blood and serum proteins blood group antigens; receptors, receptor-associated proteins, growth hormones, growth hormone receptors, T-cell receptors; neurotrophic factors, neurotrophins, relaxins, interferons, interleukins, viral antigens, lipoproteins, integrins, rheumatoid factors, immunotoxins, surface membrane proteins, transport proteins, homing receptors, addressins, regulatory proteins, and immunoadhesins.

In some embodiments, biomolecules of interest bind to one of more of the following, alone or in any combination: CD proteins including but not limited to CD3, CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvIII, cell adhesion molecules, for example, LFA-1, Mol, p150,95, VLA-4, ICAM-1, VCAM, and alpha v/beta 3 integrin, growth factors, including but not limited to, for example, vascular endothelial growth factor (“VEGF”); VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors, insulins and insulin-related proteins, including but not limited to insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF, GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; neurotrophic factors, including but not limited to, bone-derived neurotrophic factor (BDNF) and neurotrophin-3, -4, -5, or -6 (NT-3, NT-4, NT-5, or NT-6); relaxin A-chain, relaxin B-chain, and prorelaxin, interferons, including for example, interferon-alpha, -beta, and -gamma, interleukins (ILs), e.g., IL-1 to IL-10, IL-12, IL-15, IL-17, IL-23, IL-12/IL-23, IL-2Ra, IL1-R1, IL-6 receptor, IL-4 receptor and/or IL-13 to the receptor, IL-13RA2, or IL-17 receptor, IL-1RAP, IL1-α, IL-1β, viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, IgKappa, ROR-1, ERBB2, mesothelin, RANTES (regulated on activation normally T-cell expressed and secreted), mouse gonadotropin-associated peptide, DNase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, PSA, addressins, regulatory proteins, immunoadhesins, antigen-binding proteins, somatotropin, CTGF, CTLA4, eotaxin-1, MUC1, CEA, c-MET, Claudin-18, GPC-3, EPHA2, FPA, LMP1, MG7, NY-ESO-1, PSCA, ganglioside GD2, ganglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, P-cadherin, NKG2D-1, programmed cell death protein 1 and ligand, PD1 and PDL1, mannose receptor/hCGβ, hepatitis-C virus, mesothelin dsFv[PE38 conjugate, Legionella pneumophila (IIy), gpA33, B7H3, IFN gamma, interferon gamma induced protein 10 (IP10), IFNAR, TALL-1, thymic stromal lymphopoietin (TSLP), proprotein convertase subtilisin/Kexin Type 9 (PCSK9), stem cell factors, Flt-3, calcitonin gene-related peptide (CGRP), OX40L, a4β7, platelet specific (platelet glycoprotein Iib/IIIb (PAC-1), transforming growth factor beta (TFGβ), Zona Pellucida sperm-binding protein 3 (ZP-3), TWEAK, platelet derived growth factor receptor alpha (PDGFRα), sclerostin, and biologically active fragments or variants of any of the foregoing.

In some embodiments, biomolecules of interest include abciximab, adalimumab, adecatumumab, aflibercept, alemtuzumab, alirocumab, anakinra, atacicept, basiliximab, belimumab, bevacizumab, biosozumab, brentuximab vedotin, brodalumab, cantuzumab mertansine, canakinumab, cetuximab, certolizumab pegol, conatumumab, daclizumab, denosumab, eculizumab, edrecolomab, efalizumab, epratuzumab, etanercept, evolocumab, galiximab, ganitumab, gemtuzumab, golimumab, ibritumomab tiuxetan, infliximab, ipilimumab, lerdelimumab, lumiliximab, lxdkizumab, mapatumumab, motesanib diphosphate, muromonab-CD3, natalizumab, nesiritide, nimotuzumab, nivolumab, ocrelizumab, ofatumumab, omalizumab, oprelvekin, palivizumab, panitumumab, pembrolizumab, pertuzumab, pexelizumab, ranibizumab, rilotumumab, rituximab, romiplostim, romosozumab, sargamostim, tocilizumab, tositumomab, trastuzumab, ustekinumab, vedolizumab, visilizumab, volociximab, zanolimumab, zalutumumab, and biosimilars of any of the foregoing.

In some embodiments, biomolecules of interest may include blinatumomab, catumaxomab, ertumaxomab, solitomab, targomiRs, lutikizumab (ABT981), vanucizumab (RG7221), remtolumab (ABT122), ozoralixumab (ATN103), floteuzmab (MGD006), pasotuxizumab (AMG112, MT112), lymphomun (FBTA05), (ATN-103), AMG211 (MT111, Medi-1565), AMG330, AMG420 (B1836909), AMG-110 (MT110), MDX-447, TF2, rM28, HER2Bi-aATC, GD2Bi-aATC, MGD006, MGD007, MGD009, MGD010, MGD011 (JNJ64052781), IMCgp100, indium-labeled IMP-205, xm734, LY3164530, OMP-305BB3, REGN1979, COV322, ABT112, ABT165, RG-6013 (ACE910), RG7597 (MEDH7945A), RG7802, RG7813(RO6895882), RG7386, BITS7201A (RG7990), RG7716, BFKF8488A (RG7992), MCLA-128, MM-111, MM141, MOR209/ES414, MSB0010841, ALX-0061, ALX0761, ALX0141; BII034020, AFM13, AFM11, SAR156597, FBTA05, PF06671008, GSK2434735, MEDI3902, MEDI0700, MEDI7352, as well as the molecules or variants or analogs thereof and biosimilars of any of the foregoing.

Biomolecules of interest according to the disclosure encompass all of the foregoing and further include antibodies comprising 1, 2, 3, 4, 5, or 6 of the complementarity determining regions (CDRs) of any of the aforementioned antibodies. Also included are variants that comprise a region that is 70% or more, especially 80% or more, more especially 90% or more, yet more especially 95% or more, particularly 97% or more, more particularly 98% or more, yet more particularly 99% or more identical in amino acid sequence to a reference amino acid sequence of a biomolecule of interest in the form of a protein. Identity in this regard can be determined using a variety of well-known and readily available amino acid sequence analysis software. Preferred software includes those that implement the Smith-Waterman algorithms, considered a satisfactory solution to the problem of searching and aligning sequences. Other algorithms also may be employed, particularly where speed is an important consideration. Commonly employed programs for alignment and homology matching of DNAs, RNAs, and polypeptides that can be used in this regard include FASTA, TFASTA, BLASTN, BLASTP, BLASTX, TBLASTN, PROSRCH, BLAZE, and MPSRCH, the latter being an implementation of the Smith-Waterman algorithm for execution on massively parallel processors made by MasPar.

Chimeric antigen receptors incorporate one or more costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016). Suitable costimulatory domains can be derived from, among other sources, CD28, CD28T, OX40, 4-1BB/CD137, CD2, CD3 (alpha, beta, delta, epsilon, gamma, zeta), CD4, CD5, CD7, CD8, CD9, CD16, CD22, CD27, CD30, CD 33, CD37, CD40, CD45, CD64, CD80, CD86, CD134, CD137, CD154, PD-1, ICOS, lymphocyte function-associated antigen-1 (LFA-1 (CDI la/CD18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNF, TNFr, integrin, signaling lymphocytic activation molecule, BTLA, Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CDI-Id, ITGAE, CD103, ITGAL, CDI-Ia, LFA-1, ITGAM, CDI-Ib, ITGAX, CDI-Ic, ITGBI, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, 41-BB, GADS, SLP-76, PAG/Cbp, CD19a, CD83 ligand, or fragments or combinations thereof. The costimulatory domain can comprise one or more of an extracellular portion, a transmembrane portion, and an intracellular portion.

Due to the polymeric nature of anionic impurities such as PVS, these compounds can be removed by flocculation using charged particles, charged nano-particles, cationic polymers, mixed mode cationic polymers, smart polymers, and the like. PVS can be precipitated and then removed by settling or filtration using flocculants. For example, an MES buffer containing PVS can be exposed to Clarisolve® mPAA (a cationic Smart Polymer) which can subsequently be removed with the addition of the stimulus to precipitate the polymer.

The following Examples disclose functional embodiments of a method for detecting and removing polymeric anionic impurities, such as polyvinyl sulfonate (PVS), from protein and buffer solutions. In some instances, the reduction, but not complete removal, of PVS is beneficial and adequate to achieve adequate quality. In some cases, these methods are used to detect and remove anionic flocculants and residual anionic flocculants. The removal of PVS can be accomplished with anion exchange media such as chromatography resins, ion exchange resins, depth filters, synthetic depth filters, charged filters, membrane chromatography devices, mixed mode resins, and combinations thereof.

### EXAMPLES

### Example 1

### Quantification of Polyvinyl Sulfonate

Polyvinyl sulfonate concentrations were measured using a qPCR assay for DNA quantification and dilution series monitoring DNA spike recovery. The qPCR DNA assay is described in the following paragraphs.

The TaqMan™ qPCR method for residual host cell DNA quantitation has been described (Verardo et al., Biotechnol. Prog. 28:428-434 (2012)), incorporated herein in relevant part. Briefly, all test samples were diluted, if necessary, to the desired protein concentration or desired volume, as indicated, in nuclease-free water and digested with Proteinase K (Promega) at 60° C. for 2-24 hours. DNA was extracted from samples using standard chaotropic salt (sodium iodide) and alcohol precipitation protocols. Extracted DNA pellets were resuspended in nuclease-free water and the entire volume of recovered DNA was measured by qPCR total DNA analysis using the ABI QuantStudio 7 running SDS software (version 4.1). Primers were designed to amplify a CHO-cell specific repetitive DNA sequence, and a specific probe was designed to anneal between them. Forward primer Sequence: 5′ GAA ATC GGG CTG CCT GAG T 3′ (SEQ ID NO: 2); reverse primer sequence: 5′ ACC ATC CCC GAA CGA CTT C 3′ (SEQ ID NO: 3);TaqMaeprobeSequence: 5′ <FAM>CC GAG TGC GGG TGT GGT TT<TAM> 3′ (SEQ ID NO: 4). The probe is labeled with the fluorescent reporter dye FAM (6-carboxyflourescein) at its 5′ end and the quencher dye TAMRA (6-carboxytetramethylrhodamine; TAM) at its 3′ end. Standard reaction cycling conditions were utilized (50° C. for 2 minutes, 95° C. for 10 minutes, followed by 40 cycles of 95° C. for 15 seconds, and 60° C. for 1 minute). Reactions were performed in 96-well plates with 50 μL reaction volumes using Taqman® Universal PCR Master Mix (Applied Biosystems). Analysis was performed using automatic baseline settings with relative threshold values set to fall in the exponential range of amplification plots for each gene target. A standard curve of known quantities of genomic DNA isolated from the CHO host cells is used to correlate the level of standard curve fluorescence to concentrations of DNA in the original sample. Measured DNA quantities were converted to units of pg DNA/mg of sample or as otherwise indicated. All quantities were measured with duplicate or triplicate sampling and mean values were calculated.

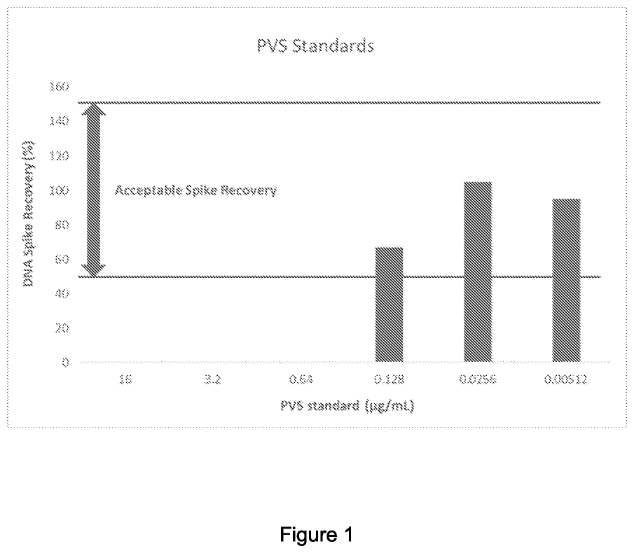

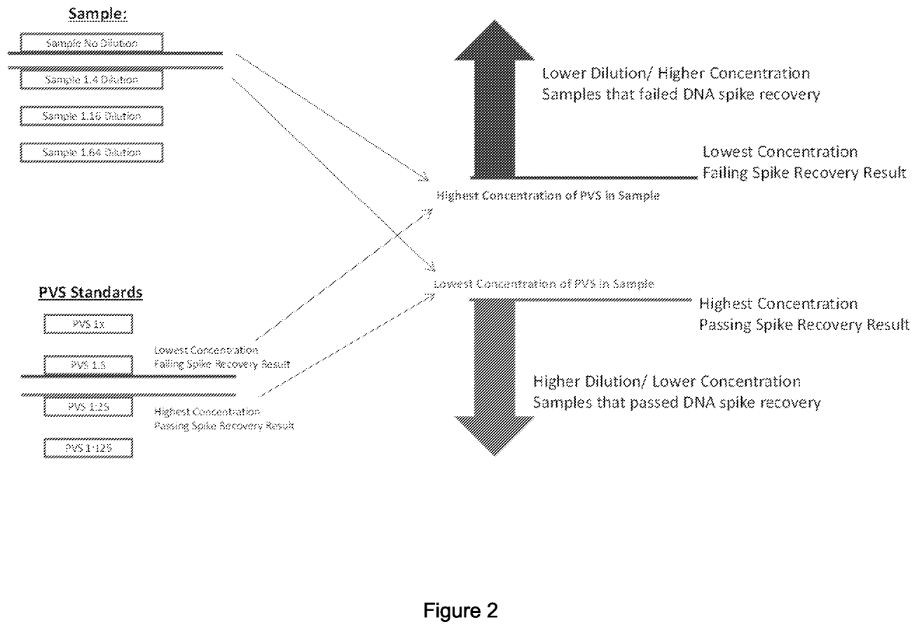

Samples were analyzed for PVS by observing qPCR assay inhibition in a sample and PVS standards dilution series (see FIG. 1). A 100 pg DNA spike was used as a positive DNA control with acceptable spike recovery between 50% to 150%. Negative controls consisting of double-deionized water showed 100% recovery. The DNA spike recovery was assessed against the PVS standards to determine the “minimum inhibiting concentration” and “maximum non-inhibiting concentration” (see FIG. 2). The minimum inhibiting concentration is the lowest concentration of sample or PVS which exhibits DNA assay interference (i.e., a failing spike recovery). This value essentially provides the “worst case” inhibitor concentration for a given sample. The maximum non-inhibiting concentration is the highest concentration with a passing DNA spike recovery, essentially providing the lowest measurable PVS quantity. For simplicity, the average PVS concentration between the minimum inhibiting and maximum non-inhibiting concentrations was used in the following analysis. In some assessments, it may be important to use the worst-case concentration. Allowable process ranges can be adjusted for the full range of measured concentrations as shown in Example 2.

### Example 2

### Removal of Polyvinyl Sulfonate from a Buffer Solution Using an Anion Exchange Membrane

A 100 mM MES buffer pH 6 was made using 21.51 g/L of MES Hydrate followed by titration with 1 M sodium hydroxide. An anion exchange membrane (0.2 μm charged nylon filter with a 2.8 cm² frontal area; Posidyne® filter) was flushed with 10 mL of de-ionized (DI) water. The 100 mM MES solution was then flushed through the AEX membrane (10 mL) and collected. The anion exchange (AEX) flow-through pool and MES buffer load material (prior to AEX) were submitted for PVS quantification via DNA assay dilution series inhibition. As expected, the load material had no DNA spike recovery (0%) due to the presence of PVS. The results are shown in Table 1. The volumetric transition for DNA spike recovery was identical for all three replicates. Therefore, the PVS removal calculated for the replicates is identical. In the MES lots used to characterize these AEX media, a 100-fold variability in PVS levels was observed. The effective membrane-loading level depends on the incoming PVS concentration (i.e., the PVS challenge on the membrane). The “PVS Removal Loading Range” is calculated by using the worst-case (maximum observed MES PVS level) to set the lower loading and the best-case (lowest observed MES PVS level) to set the upper loading level for 100 mM MES buffer solutions. The possible loading range is very large, demonstrating that charged nylon membrane sizing is dependent on the incoming PVS impurity level.

TABLE 1
Removal of PVS with an anion exchange membrane

| | | | Average | Upper | Lower | PVS Removal |
|---|---|---|---|---|---|---|
| Test | | DNA Spike | PVS | Range PVS | Range PVS | Loading |
| Run | Buffer | Recovery | Removal | Removal | Removal | Range |
| Run 1 | 100 mM | 95% | 0.53 g/M² | 0.80 g/M² | 0.26 g/M² | 10 to 10,000 L/M² |
| | MES pH 6.0 | | | | | |
| Run 2 | 100 mM | 127% | 0.53 g/M³ | 0.80 g/M² | 0.26 g/M² | 10 to 10,000 L/M² |
| | MES pH 6.0 | | | | | |
| Run 3 | 100 mM | 130% | 0.53 g/M² | 0.80 g/M² | 0.26 g/M² | 10 to 10,000 L/M² |
| | MES pH 6.0 | | | | | |

The surface area of porous media, such as membranes, is approximately proportional to the pore size. In Table 2, the preferred loading range is estimated for a smaller, 0.1 μm pore-size Posidyne filter using the assumption that the surface area and binding capacity is twice that of the 0.2 μm Posidyne filter. These loading levels are expected to provide robust removal of PVS during typical buffer preparation operations.

TABLE 2
Preferred Posidyne filter loading levels for PVS removal

| Filter | 100 mM MES Buffer Loading |
|---|---|
| 0.2 μm Posidyne filter | 30-400 L/M² |
| 0.1 μm Posidyne filter | 60-800 L/M² |

### Example 3

### Removal of Polyvinyl Sulfonate from a Buffer Solution Using a Synthetic Depth Filter

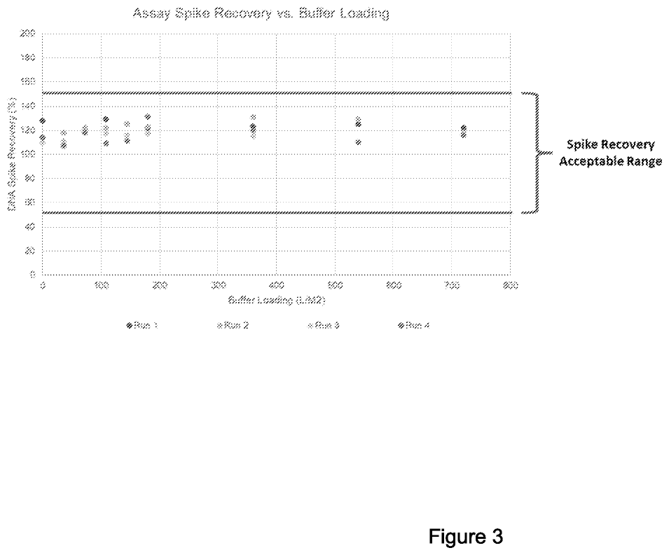

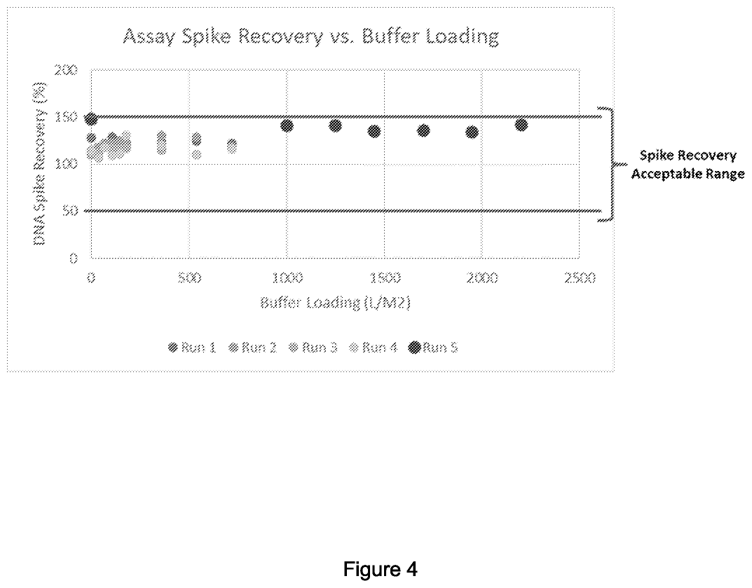

A 100 mM MES buffer pH 6 was made using 21.51 g/L of MES Hydrate followed by titration with 1 M sodium hydroxide (designated Run #1 and #2). A second MES sample was prepared in an identical manner, but with the addition of 2.04 g/L sodium chloride (targeting 35 mM NaCl, Run #3 and #4). A synthetic anion exchange depth filter (Hybrid Purifier® with a 2.5 cm² frontal area) was flushed with 90 mL of DI water. The 100 mM MES solutions were then flushed through the AEX synthetic depth filter (1800 mL) and collected. The AEX flow-through pool and MES buffer load material (prior to AEX synthetic depth filter) were submitted for PVS quantification via DNA assay dilution series inhibition. As expected, the load material had no DNA spike recovery (0%) due to the presence of PVS. The results are shown in FIGS. 3 and 4, and in Table 3. The DNA spike recovery was identical for both replicates for both conditions, and break-through of PVS at inhibitory levels was not observed. The “PVS Removal Loading Range” is calculated by using the worst-case (maximum observed MES PVS level) to set the lower loading and the best-case (lowest observed MES PVS level) to set the upper loading level for 100 mM MES buffer solutions.

TABLE 3
Results of PVS removal using a synthetic depth filter

| | | DNA | | |
|---|---|---|---|---|
| Test | | Spike | PVS | PVS Removal |
| Run | Buffer | Recovery | Removal | Loading Range* |
| Run 1 | 100 mM MES | 122% | ≥5 to | ≥200 to 25,000 L/M² |
| | pH 6.0 | | 16 g/M² | |
| Run 2 | 100 mM MES | 118% | ≥5 to | ≥200 to 25,000 L/M² |
| | pH 6.0 | | 16 g/M² | |
| Run 3 | 100 mM MES | 118% | ≥5 to | ≥200 to 25,000 L/M² |
| | pH 6.0 | | 16 g/M² | |
| | 35 mM NaCl | | | |
| Run 4 | 100 mM MES | 116% | ≥5 to | ≥200 to 25,000 L/M² |
| | pH 6.0 | | 16 g/M² | |
| | 35 mM NaCl | | | |
| Run 5 | 100 mM MES | 140% | ≥16 to | ≥600 to 25,000 L/M² |
| | pH 6.0 | | 49 g/M² | |

*Upper boundary limits calculated were in some cases >600,000 L/M² for MES buffers with lower PVS concentrations. The maximum value of 25,000 L/M² is reported as the practical limit for filtration throughput accounting for process time and other considerations.

### Example 4

### Removal of Polyvinyl Sulfonate from a Buffer Solution Using a Depth Filter

A 100 mM MES buffer pH 6 was made using 21.51 g/L of MES Hydrate followed by titration with 1 M sodium hydroxide. A depth filter with positive charge (Viresolve Pre-filter (VPF), 5 cm² frontal area) was flushed with 10 mL of DI water. The 100 mM MES solution was then flushed through the depth filter (10 mL) and collected. As expected, the load material had no DNA spike recovery (0%) due to the presence of PVS. The depth filter pool and MES buffer load material were submitted for PVS quantification via DNA assay dilution series inhibition. The results are shown in Table 4.

TABLE 4
Preferred Depth Filter loading levels for PVS removal

| Filter | 100 mM MES Buffer Loading |
|---|---|
| Viresolve Prefilter (VPF) | 15-200 L/M² |

### Example 5

Removal of Polyvinyl Sulfonate from a Buffer Solution Using an Anion Exchange Chromatography Media

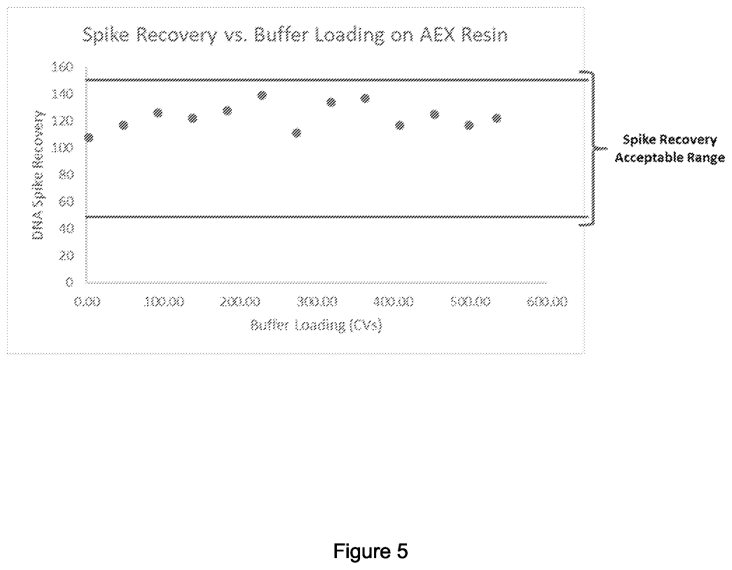

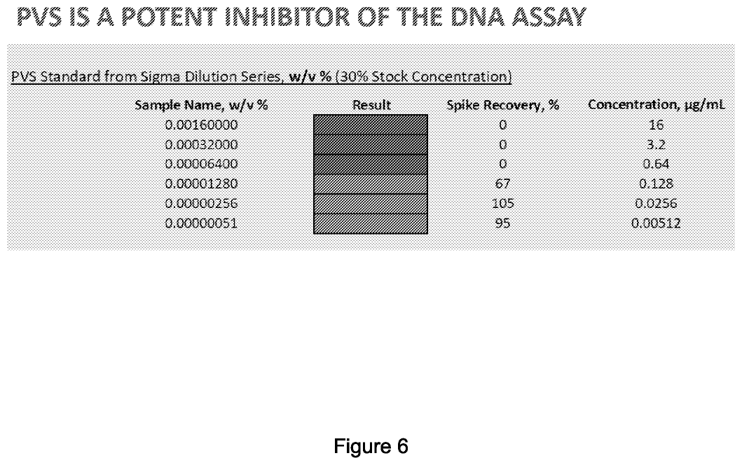

A 100 mM MES buffer pH 6 was made using 21.51 g/L of MES Hydrate followed by titration with 1 M sodium hydroxide. An anion exchange (AEX) resin with positive charge (Q-Capto™ ImpRes, 10 mL Pre-packed Column) was flushed with 30 mL DI water. The 100 mM MES solution was then flushed through the column (5370 mL) and collected. The AEX pool, fractions, and MES buffer load material were submitted for PVS quantification via DNA assay dilution series inhibition. The results are shown in FIG. 5 and Table 5. As expected, the load material had no DNA spike recovery (0%) due to the presence of PVS. In a second experiment to determine the maximum PVS binding capacity, the same AEX resin (Q-Capto™ ImpRes, 5 mL Hi-Screen Column) was flushed with 14 mL of DI water. The 100 mM MES solution was then flushed through the column (500 mL), the column washed with 20 mL of DI water and then the bound contents eluted with 3 M sodium chloride and collected. The PVS was quantified with a dilution series and the results are shown in Table 5.

TABLE 5
Preferred loading for PVS removal and Maximum
PVS binding capacity of an AEX resin

| Charged | | |
|---|---|---|
| Resin | 100 mM MES Buffer Loading | Maximum Binding Capacity |
| Q-ImpRes | ≥500 L buffer/L resin | 20-13,000 μg/mL Resin |

### Example 6

### Removal of Polyvinyl Sulfonate from a Protein Containing Solution Using an Anion Exchange Media

Several anion exchange (AEX) media were tested for the flow-through removal of PVS from a solution containing a fusion protein (protein of interest) with an isoelectric point of 8.8. All the media were flushed with DI water and then equilibrated to the test pH with 10 mL of the following buffers: 100 mM acetate pH 4.2, 25 mM Tris pH 7.4, and 25 mM Tris pH 8.0. The protein-containing solution (about 1 mg/mL protein of interest) was conditioned with a concentrated form of the target buffers to achieve the target pH and buffer concentrations shown in Table 6. The conditioned protein solution was then loaded to 30 mg protein/mL media in flow-through mode. The flow-through pool was collected and submitted for DNA assay testing. As expected, the load material had no DNA spike recovery (0%) due to the presence of PVS. At pH 4.2, no significant PVS removal was observed, resulting in DNA spike recovery failure. Although not wishing to be bound by theory, this is likely due to stronger binding between the protein of interest and the polymer inhibitor, due to the high net positive charge on the protein (pH much less than protein pl). The PVS complexed to the protein of interest greatly decreased removal in flow-through mode. Significant PVS removal was observed at pH 7.4 and pH 8.0. Again not wishing to be bound by theory, these results are likely due to a reduction in the net positive charge on the protein of interest. This effectively improves the availability of the PVS to bind to the anion exchange media (i.e., less strongly complexed to the protein of interest). The capacity in this experiment was similar for the charged membrane (Posidyne filter), a pure AEX media (Q-Sepharose Fast Flow), and a mixed mode resin with AEX functionality (Capto™ Adhere). The PVS capacity was similar in all cases studied at approximately 1.5 μg/mL of media. These data demonstrates that anion exchange media or anion exchange-containing media separate PVS from protein solution when the pH is within 1 to 2 pH units of the protein's isoelectric point.

TABLE 6
Conditions and Results for PVS removal from a protein containing solution.

| | | | | Protein | PVS | DNA |
|---|---|---|---|---|---|---|
| Protein | | | | Solution | Capacity | Assay |
| Containing | | | Anion Exchange | Loading | (μg/mL | Spike |
| Solution | Buffer | pH | Media | (mg/ml) | media) | Recovery |
| Fusion | 100 mM | 4.2 | 0.2 μm Posidyne Filter | 30 | Not | Failed |
| Protein | Acetate | | Q-Sepharose Fast Flow | 30 | Determined | Failed |
| | | | Capto Adhere | 30 | | Failed |
| | 25 mM | 7.4 | 0.2 μm Posidyne Filter | 30 | ~1.5 | Pass |
| | Tris | | Q-Sepharose Fast Flow | 30 | ~1.5 | Pass |
| | | | Capto Adhere | 30 | ~1.5 | Pass |
| | 25 mM | 8.0 | 0.2 μm Posidyne Filter | 30 | ~1.5 | Pass |
| | Tris | | Q-Sepharose Fast Flow | 30 | ~1.5 | Pass |
| | | | Capto Adhere | 30 | ~1.5 | Pass |

~: about

### Example 7

### Determination of Polyvinyl Sulfonate Binding Capacity Using Multi Modal Chromatography Media

In this experiment, PVS spiking at levels far beyond those expected in a typical downstream purification platform were tested to establish PVS binding capacity. Typical process conditions of 100 mM MES buffer at pH 6 and 100 mM MES buffer with 200 mM sodium chloride at pH 6 were tested. In addition, PVS capacity at higher sodium chloride concentrations was determined (400 mM NaCl) to represent a worst-case buffer condition.

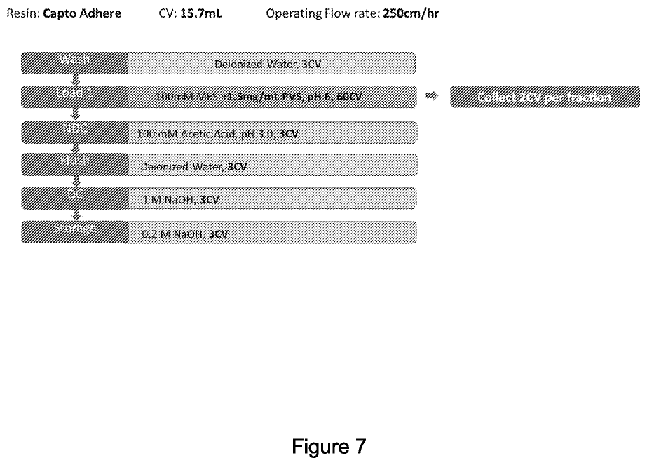

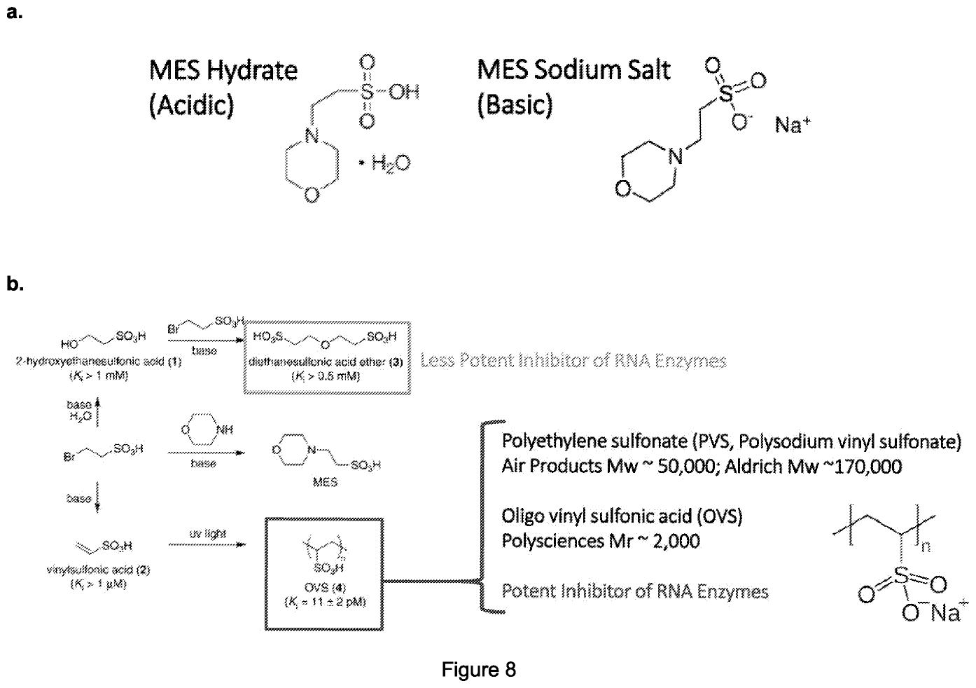

The PVS binding capacity of a Capto™ Adhere mixed mode chromatography (MMC) resin (Cytiva) was determined at lab scale. A 0.66 cm×20 cm column (15.7 mL resin) was challenged with three solutions spiked with a 30% PVS standard to achieve a PVS concentration of 1.5 mg/mL (Table 7). The column was tested per the procedure outlined in FIG. 7. MES buffer was made as described above to achieve the target buffer concentration and pH. After flushing the column with de-ionized (DI) water, PVS-spiked buffer was loaded onto the column at 250 cm/hr. Fractions were collected every 2 column volumes (CVs) for 60 CVs. The first seven fractions and pools of fractions 7-15, 16-24, and 25-30 of each buffer condition were then tested to quantify PVS levels. Each fraction or pool was measured in triplicate.

TABLE 7
Experiment design for PVS capacity determination

| | Load 30% | Replicates | |
|---|---|---|---|
| | PVS Spike | (per | |
| Buffer Solution | (mg/mL) | sample) | Comments |
| 100 mM MES pH 6.0 | 1.5* | 3 | Submitted Pools |
| | | | for fractions |
| | | | F7-F15 and later |
| 100 mM MES, 200 mM | 1.5* | 3 | Submitted Pools |
| sodium chloride, | | | for fractions |
| pH 6.0 | | | F7-F15 and later |
| 100 mM MES, 400 mM | 1.5* | 3 | Submitted Pools |
| sodium chloride, | | | for fractions |
| pH 6.0 | | | F7-F15 and later |

*PVS level was estimated using PVS standard curves..

Capto™ Adhere mixed-mode resin uses anion exchange and hydrophobic ligands to support two binding modes. Increasing NaCl represents reduced anion exchange binding of PVS and increased hydrophobic interaction. PVS capacity at higher sodium chloride concentrations was determined (400 mM NaCl) to represent a worst-case buffer condition. The results of the DNA qPCR spike recovery assay are shown in Table 8. A passing DNA spike recovery result demonstrated a concentration of PVS acceptable for the quantification of DNA using current DNA assay procedures. Using a PVS standard curve, the PVS concentration for a passing and failing result were estimated. The PVS binding capacity of the resin was determined by the amount of PVS bound up to the PVS break-through (first failing DNA spike recovery result) and shown in Table 8. The binding capacities of the mixed-mode resin for the corresponding chromatography fractions are also shown in Table 8 (second column). The 400 mM NaCl buffer condition represents a worst-case scenario wherein ionic interactions are reduced due to increased salt concentrations. This observation is consistent with polyvinyl sulfonate's highly charged structure, with each polymeric repeat unit possessing a negative sulfonate group.

TABLE 8
DNA qPCR Assay spike recovery assessment for Capto ™ Adhere
PVS removal studies

| | | DNA qPCR Assay DNA Spike | | |
|---|---|---|---|---|
| | | Recovery Result | | |
| | | | 100 mM | 100 mM |
| | PVS Binding | | MES, | MES, |
| | Capacity | | 200 mM | 400 mM |
| | (mg PVS/ | 100 mM MES | NaCl, | NaCl, |
| Fractions | mL resin) | pH 6.0 | pH 6.0 | pH 6.0 |
| F1 | N/A | N/A* | N/A* | N/A* |
| F2 | 3 | Pass | Pass | Pass |
| F3 | 6 | Pass | Pass | Pass |
| F4 | 9 | Pass | Pass | Fail |
| F5 | 12 | Pass | Pass | Fail |
| F6 | 15 | Pass | Pass | Fail |
| F7-F15 | 18 | Fail | Fail | Fail |
| F16-F24 | 21 | Fail | Fail | Fail |
| F25-F30 | 48 | Fail | Fail | Fail |

N/A—Not Applicable, the first fraction was determined to be non-representative due to buffer exchange effects, subsequent fractions do not show qPCR interference.

The DNA assay spike recovery was determined for the flow-through fractions. Passing fractions demonstrated acceptable PVS clearance and the first failing sample defined the PVS binding capacity for the Capto™ Adhere resin. For example, the standard condition samples (100 mM MES pH 6; 100 mM MES, 200 mM NaCl, pH 6) showed acceptable clearance for loadings up to Fraction 7. Fraction 7 represents a PVS loading of 15 mg PVS/mL resin and loading PVS above this level caused consistent spike recovery interference (pools of fractions 7-30 all showed failing spike recovery). Analysis of the worst-case, 400 mM NaCl condition showed spike recovery interference in fraction 4. Thus, the worst-case binding capacity was 9 mg PVS/mL resin. Therefore, a binding capacity of 9 to 15 mg PVS/mL resin was observed for mixed mode resin.

### Example 8

### Titration Methods for Detecting and Removing Polyanions

Nine lots of commercial MES buffer were obtained and subjected to analysis using the titration method for detecting and measuring PVS disclosed herein. Comparative assessments of these lots of MES buffer were performed using the disclosed titration method of detecting and measuring PVS and using qPCR. As the experimental data shows, the method is capable of sensitive detection of low levels of PVS and accurately and precisely detects lot-to-lot variation in PVS levels. Such analyses revealed a commercial lot of MES buffer (Lot #I) containing a markedly high level of PVS, consistent with observations of lot-to-lot variability in buffer-associated inhibition of host cell nucleic acid contamination of biologic samples using PCR (e.g., qPCR).

The data provided in this Example and in Example 9 establish that the titration method of detecting and measuring PVS in samples using a polycationic compound such as hexadimethrine bromide (i.e., HDBr) is highly selective for PVS over MES, with a K_{a, PVS}>>Kₐ, MES. The results disclosed herein reveal that the disclosed titration method is repeatable (precise) and capable of detecting low levels of polyanions, e.g., PVS, in Good's buffers such as MES, with aa limit of quantitation (i.e., LOQ) of about 100-200 ng/ml.

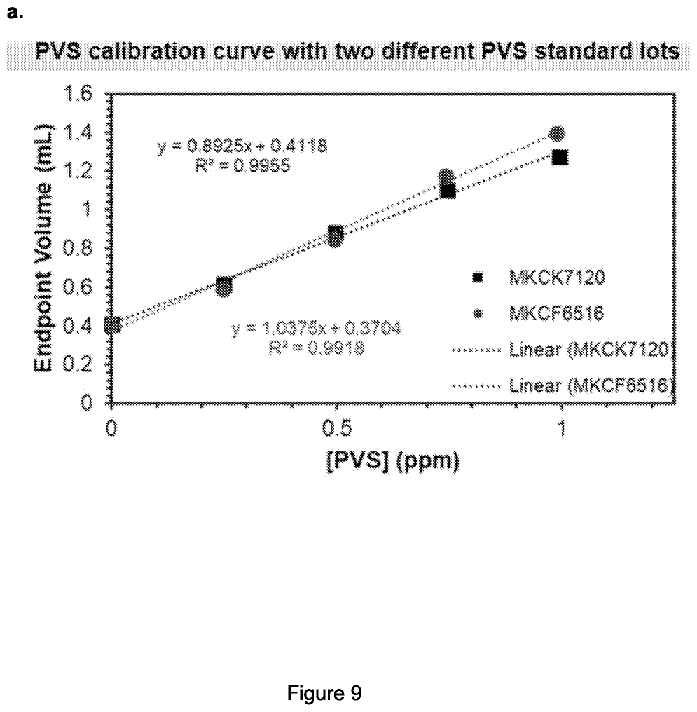

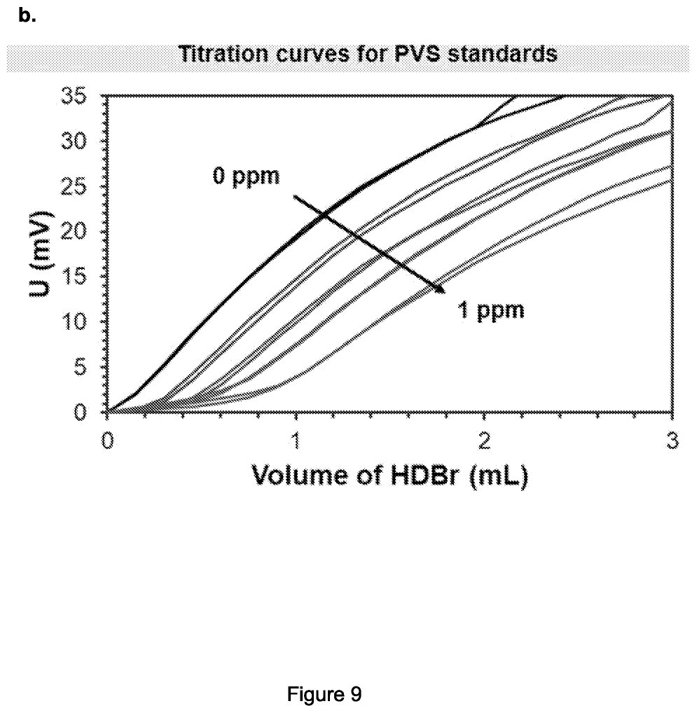

The protocol disclosed herein describes a polyelectrolyte titration approach to quantitating polyanions such as polyvinyl sulfonic acid) (PVS) in Good's buffers such as 2-(N-morpholino)ethanesulfonic acid (MES) buffer. The methodology is extendable to other Good's buffers (e.g., HEPES) produced from vinylsulfonic acid. The underlying mechanism for PVS detection is based on binding with a polycationic species such as hexadimethrine bromide (HDBr). A schematic of the binding reaction is provided in FIG. 10a. This approach exploits the high equilibrium association constant (Ka) between PVS and HDBr for selectivity over MES, a monoanion. Indeed, the Ka between a polycation and polyanion increases steeply with the number of charge sites (positively correlated to polymer molecular weight). At the endpoint of the titration, excess HDBr associates with the anionic indicator molecule, Eriochrome Black T (ECBT), producing a shift in the UV-Vis absorbance profile of the indicator (FIG. 10b). The progression of the titration can be tracked at a single wavelength (i.e., 665 nm) and related to the concentration of PVS in the sample by, for example, calculating the inflection point of the resulting sigmoidal curve, as shown in FIG. 9.

### Materials and Methods for Examples 8 and 9

In reagent preparation, assay buffers are prepared using conventional techniques to yield Buffer A comprising 50 mM sodium borate, with pH adjusted to 8.5 with hydrochloric acid, and Buffer B comprising 100 mM combined sodium carbonate and bicarbonate, formulated to produce a solution of pH 10.0. An indicator compound or dye solution such as a polyanionic indicator compound, e.g., Eriochrome Black T (ECBT; 55 wt %), serves as the indicator compound. When the indicator compound was ECBT, a solid aliquot of this material was stored at room temperature. To prepare an exemplary ECBT dye solution, 125 mg of ECBT was added to a 25 mL volumetric flask and the actual mass was recorded. The ECBT was dissolved in 25 mL de-ionized (i.e., DI) water and aliquoted into 1.6 mL or 5 mL polypropylene microcentrifuge tubes and stored at 2-8° C. until use. The polycationic compound of the disclosed methods is a titrant, and an exemplary titrant solution is made using hexadimethrine bromide (HDBr). This material is stored at 2-8° C. To prepare the solution, 18.7 mg HDBr was weighed directly into a glass vial and dissolved in 3.74 mL water to yield a 5 mg/mL stock solution. 0.05 μg/ml HDBr titrants were then prepared by 1:20 or 1:100 dilution, respectively, of the 5 mg/ml HDBr solution in 50 mM borate buffer supplemented with 0.1 mM EDTA. This solution was used as the titrant solution for the assay methods disclosed herein. The HDBr titrant solutions were prepared as 10 mL solutions in 15 mL polypropylene centrifuge tubes and stored at 2-8° C.

For the experiments involving titration methods to detect and remove biomolecules, approximately 30 wt % poly(vinylsulfonic acid) (PVS) sodium salt was purchased from Sigma-Aldrich (#278424) and Alfa Chemistry (#ACM25053274) and was diluted to prepare PVS standards of known concentration ranging from 0.1 to 20 μg/mL. 50 mM borate buffer (pH 8.5) was prepared using conventional techniques. 100 mM carbonate buffer (pH 10.0) was prepared from sodium carbonate (Sigma-Aldrich #223484) and sodium bicarbonate (Sigma-Aldrich #S6014). The carbonate and bicarbonate buffers were supplemented with about 0.1 mM ethylenediaminetetraacetic acid (EDTA; MP Biomedicals #06133713). 1,5-dimethyl-1,5-diazaundecamethylene polymethobromide (Hexadimethrine bromide; HDBr) was purchased from Sigma-Aldrich (107689) and Carbosynth (#FH165280). Eriochrome Black T (EBT or ECBT) was purchased from Sigma-Aldrich (#858390). All solutions were prepared using water that had been purified to a minimum resistivity of 18 MΩ-cm. A 100 mM solution of MES hydrate was cleared of PVS by filtration over a 0.2 μm Posidyne® filter (2.8 cm² surface area) and served as the sample blank for the experiments disclosed herein.

### Standard Preparation

A commercial poly(vinylsulfonate) (PVS) stock solution was used to prepare the assay standards (Alfa Chemistry, 25 wt %, sodium-salt, Lot# A19X05191) by performing serial dilutions of the stock solution in water. The PVS solutions in Table 9 where then spiked into 30 mM borate buffer (supplemented with 0.1 mg/mL EDTA) to prepare standards of known PVS concentrations.

TABLE 9

| | | | | Approxmate |
|---|---|---|---|---|
| PVS Stock | | | Dilution | [PVS] |
| Dilution Factor | V_{water} (mL) | V_{Standard} (mL) | used | (μg/mL) |
| 1:20 | 0.950 | 0.050 | Stock | 12500 |
| 1:400 | 0.950 | 0.050 | 1:20 | 625 |
| 1:2000 | 0.800 | 0.200 | 1:400 | 125 |
| 1:10000 | 0.800 | 0.200 | 1:2000 | 25 |

Stocks and standard solutions were stored at 2-8° C.

### Sample Preparation

100 mM solutions of MES hydrate (Lot #s I and II), pH adjusted to 7.00±0.05 were prepared as follows. 2.132 g MES hydrate were dissolved in 95 mL water, and the pH was adjusted using aqueous NaOH. The pH was measured using a conventional pH meter. Solutions were stored at 2-8° C.

### Assay Procedure

Although titration feasibility experiments were carried out using a simple protocol described below, such experiments could be automated by using a photometric titrator instrument to automate the steps described herein. UV and visible lamps of the spectrometer were warmed for at least 20 minutes prior to use by turning on the spectrometer. The spectrometer was blanked before each assay using either the standard or sample solutions. The standard cell used in the disclosed assay was a 10 mm, 1.5 mL quartz cuvette. The standard consists of PVS diluted in assay buffer. The sample is prepared by mixing 100 mM MES as an exemplary Good's buffer with assay buffer. This step is performed because the exemplary ECBT indicator compound undergoes a color change over pH values of 6-7, whereas pH values greater than 7 are above the buffer region for MES. Therefore, MES was mixed with basic buffers, i.e., A or B, as described above, to ensure that the ECBT indicator was deprotonated.

Initial experiments mixed Buffer A and MES in a 1:1 ratio. It is expected that more basic buffers (e.g., B), mixed with MES in different volumetric ratios, will increase assay performance.

Once the spectrometer was blanked, a small volume of the ECBT solution was added to the standard/sample. Initially, 995 μL standard/sample were mixed with 5 μL ECBT (5 mg/mL), yielding a final ECBT concentration of 25 μg/mL. Full wavelength absorbance scans were acquired. The standard/sample solution was titrated by adding small volumes (10-100 μL) of the 0.050 mg/mL HDBr solution to the cuvette, measuring the sample absorbance between each HDBr addition. A 200 μL pipette was used to mix the solution and the solution was allowed to stand for about 1 minute before measuring absorbance. The volume of HDBr was gradually increased over the course of the titration. For instance, small-volume (e.g., 10 μL) additions were initially performed, as the absorbance profile changed drastically early in the titration. Larger volumes were added later in the titration when the absorbance change was more significantly affected by dilution. In some instances, (e.g., for solutions with larger PVS concentrations), a more concentrated 0.25 mg/mL HDBr solution was used. The preceding steps of blanking the spectrometer, and adding a small volume of the indicator compound solution to the standard/sample were then repeated for each sample.

### Data Analysis

From UV-vis spectra, absorbance was plotted at 665 nm against the mass of HDBr added (in μg). The absorbance should be corrected for the change in solution volume to account for dilution, which was achieved by multiplying A_{665 nm} by the total solution volume (i.e., original volume of the solution [1.000 mL], plus the cumulative volume of titrant solution added).

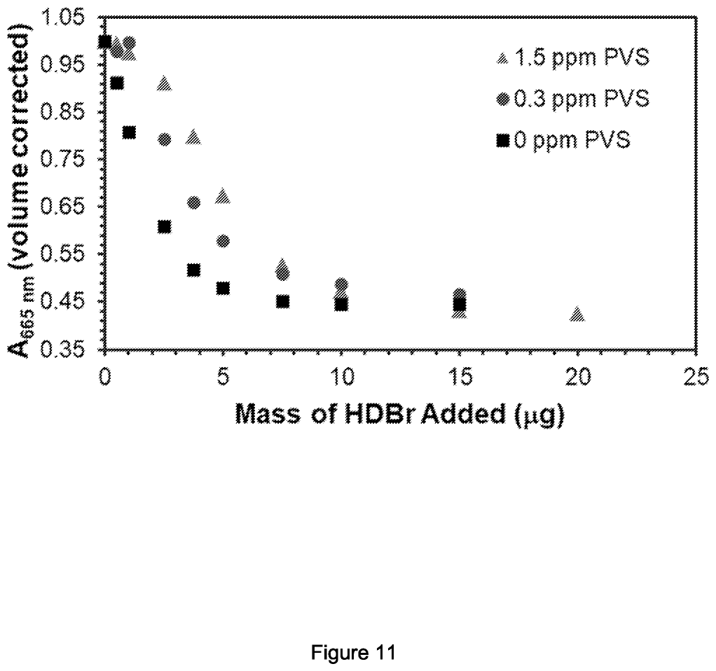

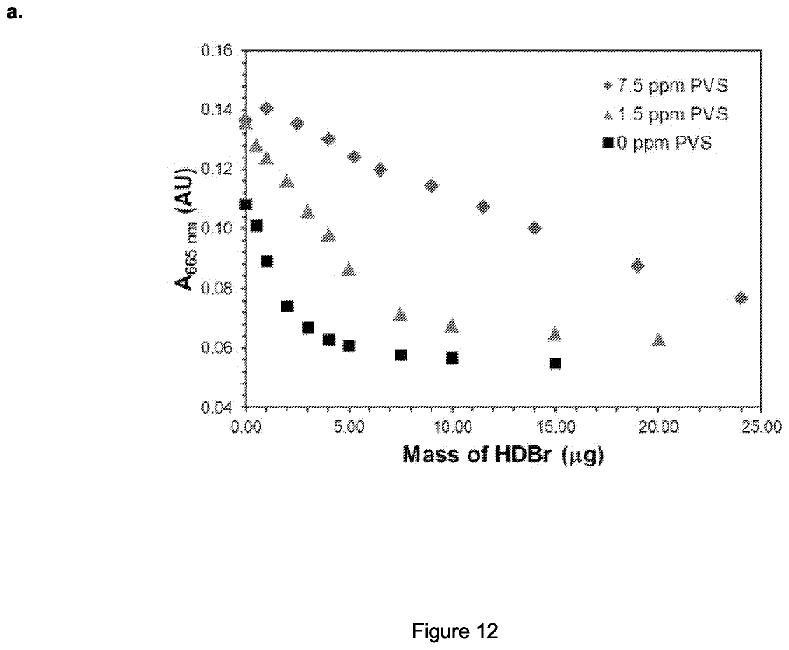

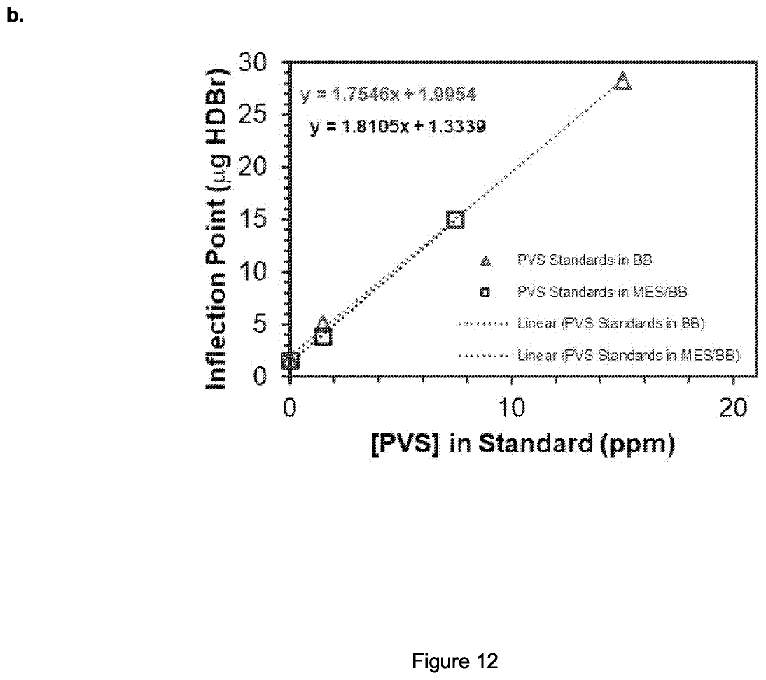

FIGS. 11 and 12 summarize the results of the assessment. FIG. 4 presents the volume-corrected solution absorbance at 665 nm with respect to the mass of HDBr titrant for assay buffer spiked at three different PVS levels.

FIG. 12a presents the volume-corrected solution absorbance at 665 nm with respect to the mass of HDBr titrant for MES matrix blank spiked at three different PVS levels. For both the 0 ppm PVS standard and the sample blank (i.e., MES blank), addition of titrant caused a precipitous initial decline in A₆₆₅, which stabilized after about 5.00 μg HDBr was added to the solution. The remaining PVS standards samples, which were prepared by spiking commercially sourced PVS into solution, required a larger amount of titrant to reach steady-state absorbance. For example, the 7.5 ppm sample (FIG. 12a) achieved stable A₆₆₅ only after more than 40 μg HDBr was added. Taken together, these data indicated clear differences in the titration curves (FIGS. 11 and 12a) related to the quantity of PVS in the sample solution. FIG. 12b summarizes this relationship by plotting the calculated inflection point for PVS standard solutions prepared in 50 mM borate buffer (pH 8.5) (green triangle) or MES that had been spiked with PVS and subsequently mixed with 50 mM borate buffer (pH 8.5) to adjust the solution pH (black squares). The slopes for the two sets of data were comparable, indicating that the presence of MES at high concentration (100 mM) did not interfere with PVS quantitation. Furthermore, these data support detection of PVS at concentrations as low as 1.5 ppm (μg/mL) in 100 mM MES solution, 0.3 ppm in assay buffer.

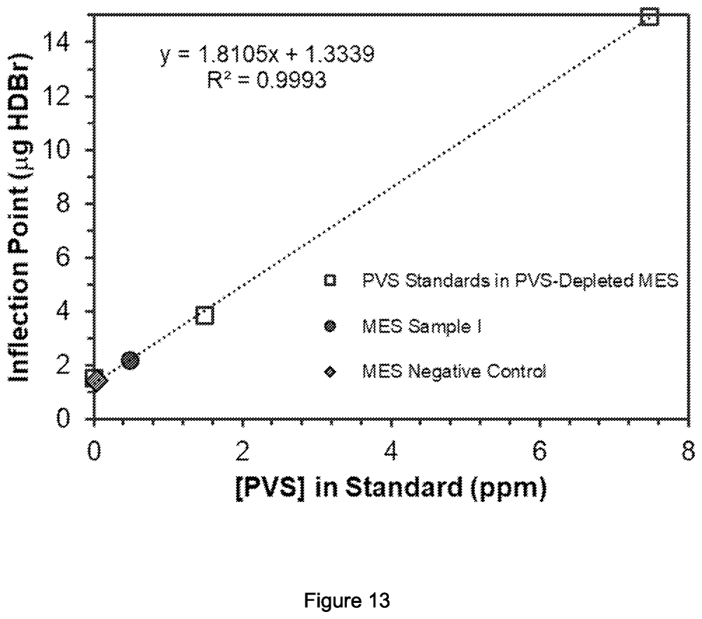

To further evaluate the performance of the titration procedure, two distinct lots of MES were evaluated alongside the PVS standards. A lot of MES hydrate (Sample I) that resulted in invalid qPCR results for several products was compared to another MES sample that had a minimal amount of PVS per qPCR assay (i.e., the same material used to generate the sample blank in FIG. 12a). The results for this assessment, shown in FIG. 13, showed a measurable amount of PVS present in MES sample I, but not in the negative control MES material, which was indistinguishable from the PVS-depleted matrix blank. These results establish that the disclosed methodology can accurately identify MES hydrate materials with unsuitable levels of PVS. Moreover, MES hydrate materials without PVS, or with intermediate levels of PVS that would not interfere with qPCR, are distinguished from the unsuitable MES materials.

### Example 9

### Automated Titration

Automated titrations of the PVS standard and MES sample solutions were carried out using a Metrohm 907 Titrando instrument equipped with an intelligent dosing drive (#2.800.0010) and a dosing unit with a 20 mL volume capacity (#6.303.2200). 100 mL standard or sample solution was supplemented with 0.8-1.7 μg/mL EBT indicator (e.g., by spiking in 0.5-1.0 mg/mL EBT stock) immediately before titration. The resulting solution was assayed by monotonically titrating the sample with HDBr in 50-150 μL volume increments. The titration progress was monitored by continuously measuring sample solution absorbance at 660 nm using an immersible photometric probe (Optrode, #6.1115.000), with the titration end point determined using the maximum dU/dV in the titration curve first derivative.

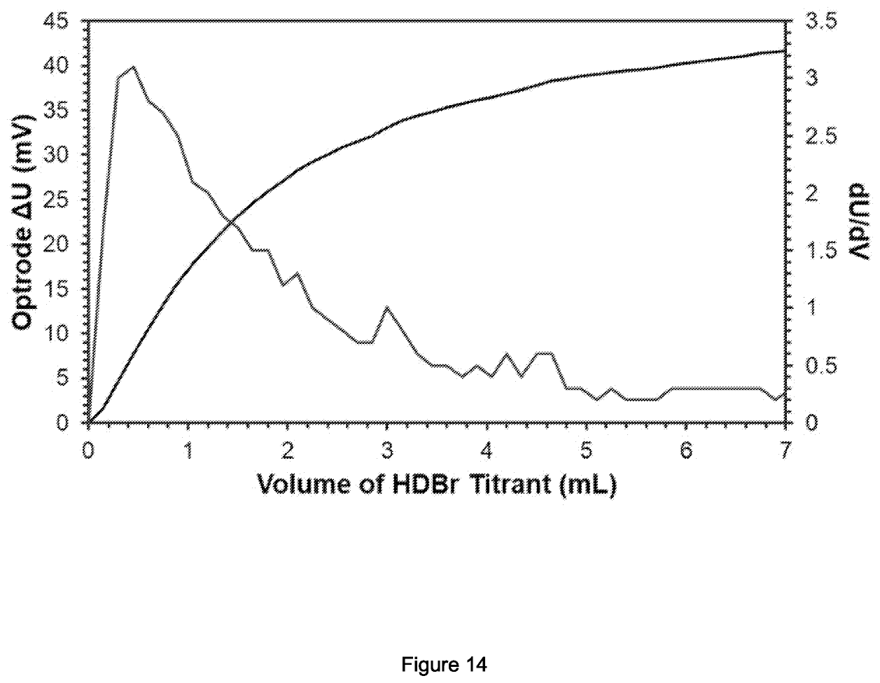

Automated PVS measurements were carried out using a 907 Titrando (Metrohm) equipped with an immersible photometric probe capable of solution absorbance measurements at a wavelength of 660 nm. The titrations were carried out by incremental dosing of 0.05-0.15 mL HDBr titrant solution. In between titrant increments, the signal from the photometric probe was allowed to stabilize prior to dosing the next titrant volume. A representative titration profile for a blank standard is presented in FIG. 14 (black trace), alongside the corresponding first derivative (red trace). The volume at which the maximum in the first derivative occurs (i.e., V_{Titrant} of approximately 0.55 mL in FIG. 14) signifies the titrant end point and is used in the determination of PVS concentration.

The pH of the sample solution plays an important role in the measurement of PVS, either by impacting the anionic charge density on the PVS analyte or indirectly by protonation of the indicator compound to form the monovalent anion (H₂In⁻), which does not undergo a change in absorbance upon complexation with HDBr. The experiments, described above in Example 8, indicated that mixing prepared MES solutions with an alkaline buffer would be a viable approach for ensuring a suitable sample pH. Use of this approach in automated titration experiments (i.e., by dissolution of MES samples at 50 mM MES in 100 mM carbonate buffer) was verified through an assessment of PVS spike recovery in MES sample solutions. For this evaluation, 10 ppm PVS stock solution was spiked at varying concentrations into sample solutions corresponding to a lot of MES hydrate (Sample H; see Table 10). This material, when assayed by titration, produced end point volumes indistinguishable from the blank standard, indicating a PVS level below the method limit of detection.

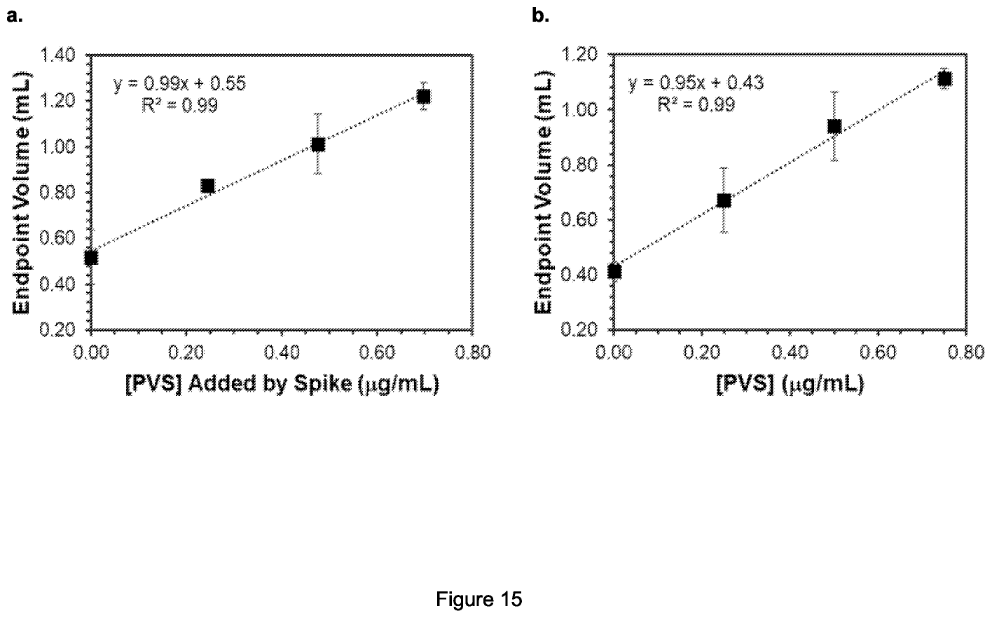

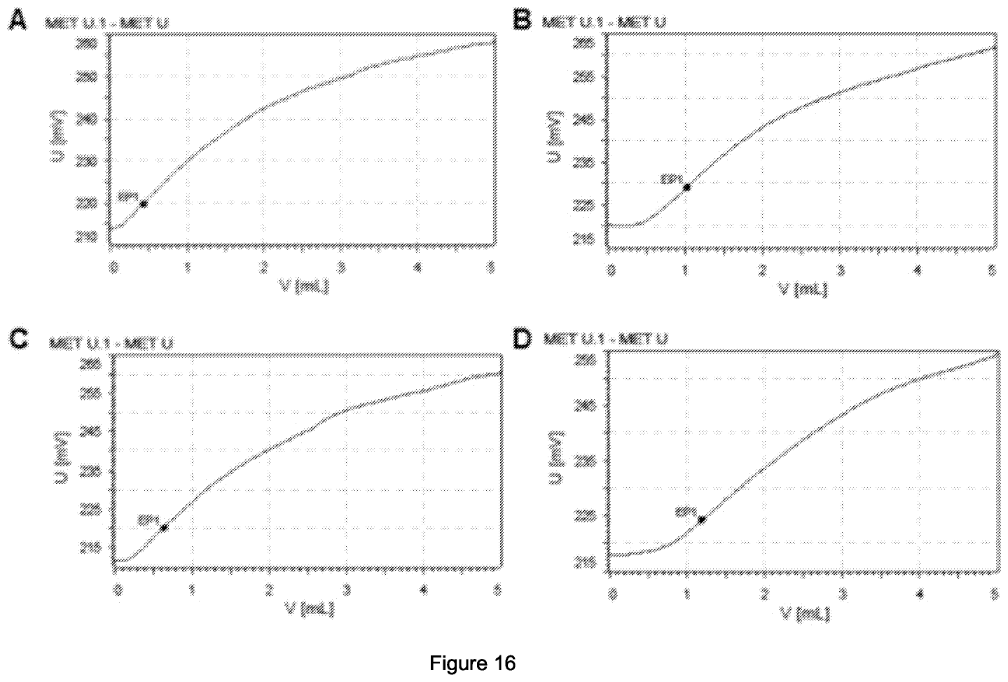

The results for the spike recovery assessment are presented in FIG. 15a, which plots the titration end point volume against the concentration of PVS at four different PVS levels (each assayed in triplicate). For comparison, results for PVS standards prepared in 100 mM carbonate buffer alone are shown in FIG. 15b. For both sets of data, linear regression between the titration end point volume and the PVS concentration resulted in similar slopes (0.99 and 0.95 mL/(μg/mL)) with appropriate linear coefficients of determination (R²=0.99). Notably, the magnitude of the y-axis intercept for the spike recovery data (0.55 mL) was larger than that for the standard curve in FIG. 15b (0.43 mL), which is likely due to a low level of PVS in MES Sample H. In addition, visual examination of the representative titration curves shown in FIG. 16 for the PVS standards (FIGS. 16A and 16B) and spike recovery samples (FIGS. 16C and 16D) showed no perceptible impact of the lower pH or the presence of 50 mM MES to the titration profile. Collectively, these results indicate no appreciable impact of the lower sample pH or the presence of 50 mM MES to measured PVS levels.

Over the course of development of the titration procedure, several MES hydrate lots were evaluated for PVS content by titration of 50 mM MES (dissolved in 100 mM carbonate buffer) with 0.10 mg/mL HDBr. The titration end points were compared to results generated for a series of PVS standard solutions. The results from these assessments are presented in Table 10. Among these samples was a lot of MES hydrate (Sample I) that caused failure of the qPCR assay for several therapeutic protein batches. Sample I had a PVS level, measured by titration, of 71±4 μg PVS per gram of MES hydrate, a value significantly greater than the PVS levels measured for any of the other samples tested, supporting the utility of the titration in screening MES materials with unsuitable levels of PVS.

TABLE 10
MES hydrate samples evaluated for PVS
during titration method development

| MES Hydrate | [PVS] in 100 mL | [PVS], Dry Basis |
|---|---|---|
| Sample | 50 mM MES (μg/mL) | (μg PVS/g MES Hydrate) |
| A^{a} | 0.21 ± 0.07 | 21 ± 7 |
| B^{a} | 0.38 ± 0.18 | 39 ± 19 |
| C^{a} | 0.26 ± 0.09 | 27 ± 9 |
| D^{b} | 0^{c} | 0^{c} |
| E^{b} | 0.14 | 14 |
| F^{b} | 0.25 | 25 |
| G^{b} | 0.04 | 4 |
| H^{b} | 0^{c} | 0^{c} |
| I^{c} | 0.71 ± 0.04 | 71 ± 4 |

^{a}Samples were evaluated in triplicate.
^{b}Samples were evaluated without replicate measurement.
^{c}Sample was below the limit of detection (LOD), generating a negative [PVS].

### Example 11

### Comparison of Detection Methods

Several methods for detecting and measuring polycations such as PVS in protein samples (e.g., biologic samples) were assessed. An ion coordination method involving induced aggregation of a reporter by PVS with turbidimetric detection is a straightforward method of low complexity, but the method failed to reliably detect lots of MES buffer having high levels of PVS. A fluorescence based method involving direct detection of aqueous PVS via fluorescence excitation and detection was another straightforward method of low complexity, but the method proved infeasible for detection of PVS. Another fluorescence-based method involved PVS-induced quenching of a fluorescent reporter molecule was more involved and did not show promise because of limited capacity to selectively detect PVS relative to MES. A method based on the physical characteristics of polycations found in Good's buffers is size exclusion chromatography with charged-aerosol detection (i.e., SEC-CAD). This method was capable of detecting PVS in MES buffers, but the method is considerably more complex than the other methods. One more ion coordination method was assessed and that method, involving polyelectrolyte complexation and titration using ultraviolet-visible wavelength absorbance detection, was found to produce unexpectedly superior results in providing accurate, precise and sensitive detection and quantitation of PVS in Good's buffers, including but not limited to the Good's buffers provided in Table 11. This method, disclosed herein as the titration method, is a straightforward method of low complexity and cost in addition to providing the benefits of accuracy, precision and sensitivity.

TABLE 11
Good's buffers

| Buffer | pKₐ | Buffer | pKₐ |
|---|---|---|---|
| MES | 6.15 | POPSO | 7.85 |
| Bis-tris methane | 6.60 | HEPPSO | 7.9 |
| ADA | 6.62 | EPS | 8.0 |
| Bis-tris propane | 6.80 | HEPPS | 8.1 |
| PIPES | 6.82 | Tricine | 8.15 |
| ACES | 6.88 | Tris | 8.2 |
| MOPSO | 6.95 | Glycinamide | 8.2 |
| Cholamine chloride | 7.10 | Glycylglycine | 8.2 |
| MOPS | 7.15 | HEPBS | 8.3 |
| BES | 7.17 | Bicine | 8.35 |
| AMPB | 8.8 | TAPS | 8.55 |
| HEPES | 7.55 | AMPB | 8.8 |
| DIPSO | 7.6 | CHES | 9.3 |
| MOBS | 7.6 | CAPSO | 9.6 |
| Acetamidoglycine | 7.7 | AMP | 9.7 |
| TAPSO | 7.6 | CAPS | 10.4 |
| TEA | 7.8 | CABS | 10.7 |

Each of the references cited herein is hereby incorporated by reference in its entirety or in relevant part, as would be apparent from the context of the citation.

It is to be understood that while the claimed subject matter has been described in conjunction with the detailed description thereof, the foregoing description is intended to illustrate and not limit the scope of that claimed subject matter, which is defined by the scope of the appended claims. Other aspects, advantages, and modifications are within the scope of the following claims.

1. A method for quantification of a polyanionic PCR inhibitor in a sample comprising:
a) Preparing a dilution series of a sample comprising at least four members;
b) Spiking each member of the dilution series with a constant amount of a template DNA distinguishable from host cell DNA;
c) Performing a PCR assay on each member of the dilution series and on the constant amount of the template DNA in the absence of any sample;
d) Generating a polyanionic inhibitor standard curve;
e) Comparing the PCR assay results of the dilution series to the PCR assay results of the constant amount of the template DNA in the absence of any sample; and
f) Identifying the concentration of polyanionic PCR inhibitor in the sample.

2. The method of claim 1 wherein the concentration of polyanionic PCR inhibitor in the sample is a range defined by the concentration of polyanionic PCR inhibitor in the least diluted member of the dilution series showing complete spike recovery and the most diluted member of the dilution series not showing complete spike recovery.

3. The method of claim 2 wherein the number of members in the dilution series is 5, 6, 7, 8, 9, 10, 12, 15, or 20, thereby narrowing the range of concentration of the polyanionic PCR inhibitor in the sample relative to the range provided in claim 2.

4. (canceled)

5. The method of claim 1 wherein the polyanionic PCR inhibitor is a sulfonate compound.

6. (canceled)

7. The method of claim 1 wherein polyvinyl sulfonate is the polyanionic PCR inhibitor used in generating the polyanionic inhibitor standard curve and the concentration of polyanionic PCR inhibitor in the sample is in units of polyvinyl sulfonate concentration equivalents.

8. A method for removing a polyanionic PCR inhibitor from a buffer solution comprising:
a) preparing a buffer solution of an acidic buffering species, a basic buffering species, or a combination thereof;
b) contacting the buffer solution with an anion exchange medium or a mixed mode resin; and
c) separating the buffer solution from the polyanionic impurity, thereby removing the polyanionic impurity from the buffer solution.

9. (canceled)

10. The method of claim 8 wherein the polyanionic impurity is a sulfonate compound.

11. (canceled)

12. The method of claim 8 wherein the buffer solution is a Good's Buffer solution.

13. (canceled)

14. (canceled)

15. The method of claim 8 further comprising adding at least one modifying compound to the buffer solution.

16. (canceled)

17. The method of claim 8 wherein the anion exchange medium is diethylaminoethyl-modified matrix, Dimethylaminoethyl-modified matrix, dimethylaminopropyl-modified matrix, polyethyleneimine-modified matrix, quaternized polyethyleneimine-modified matrix, fully quaternized amine-modified matrix, anion exchange modified diatomaceous earth containing depth filters, anion exchange membrane adsorbers, salt tolerant anion exchange membrane adsorbers, Macro-Prep 25Q, TSK-Gel Q, Poros Q, Q Sepharose Fast Flow, Q HyperD, Q Zirconia, Source 30Q, Fractogel EMD TMAE, Express-Ion Q, DEAE Sepharose Fast Flow, Poros 50 D, Fractogel EMD DEAE (M), MacroPrep DEAE Support, DEAE Ceramic HyperD 20, Toyopearl DEAE 650 M, Capto Q, Sartobind Q membrane absorber, Posidyne charged membrane, Amberlite® (polyamine)-modified matrix, Amberlite® (iminodiacetic acid)-modified matrix, Amberlite® Type I (trialkylbenzyl ammonium)-modified matrix, Amberlite® Type II (dimethyl-2-hydroxyethylbenzyl ammonium)-modified matrix, Dowex® (polyamine)-modified matrix, Dowex® Type I (trimethylbenzyl ammonium)-modified matrix, Dowex® Type II (dimethyl-2-hydroxyethylbenzyl ammonium)-modified matrix, Dowex ® (mixed bed), Capto® Adhere Anion Exchange Multi Mode, PPA Hypercel, HEA Hypercel, or Duolite® (polyamine)-modified matrix.

18. The method of claim 8 wherein the mixed mode resin is Capto® Adhere Anion Exchange Multi Mode resin, PPA Hypercel resin, or HEA Hypercel resin.

19. The method of claim 8 wherein the anion exchange medium binds up to 15 mg PVS per mL of anion exchange medium, up to 9 mg PVS per mL anion exchange medium, or up to 3 mg PVS per mL anion exchange medium.

20. (canceled)

21. (canceled)

22. The method of claim 8 wherein the mixed mode resin binds up to 15 mg PVS per mL of mixed mode resin, up to 9 mg PVS per mL of mixed mode resin, or up to 3 mg PVS per mL of mixed mode resin.

23. (canceled)

24. (canceled)

25. The method of claim 8 wherein the anion exchange medium is a polycationic compound that forms a complex with the polyanionic impurity (analyte).

26. The method of claim 25 wherein the polycationic compound is a quaternary ammonium-based polymer.

27. (canceled)

28. A method for removing a polyanionic buffer impurity from a protein solution comprising:
a) adjusting the pH of a protein solution containing an anionic buffer impurity to a pH less than the isoelectric point of the protein by no more than 4 pH units;
b) contacting the protein solution with an anion exchange medium or a mixed mode resin; and
c) separating the protein from the anionic buffer impurity.

29. (canceled)

30. The method of claim 28 wherein the pH is adjusted to be lower than the isoelectric point of the protein by no more than 2 pH units.

31. The method of claim 28 wherein the anion exchange medium is diethylaminoethyl-modified matrix, Dimethylaminoethyl-modified matrix, dimethylaminopropyl-modified matrix, polyethyleneimine-modified matrix, quaternized polyethyleneimine-modified matrix, fully quaternized amine-modified matrix, an anion exchange-modified diatomaceous earth-containing depth filter, an anion exchange membrane adsorber, a salt-tolerant anion exchange membrane adsorber, Macro-Prep 25Q, TSI-Gel Q, Poros Q, Q Sepharose Fast Flow, Q HyperD, Q Zirconia, Source 30Q, Fractogel EMD TMAE, Express-Ion Q, DEAE Sepharose Fast Flow, Poros 50 D, Fractogel EMD DEAE (M), MacroPrep DEAE Support, DEAE Ceramic HyperD 20, Toyopearl DEAE 650 M, Capto Q, Sartobind Q membrane absorber, Posidyne charged membrane, Amberlite® (polyamine)-modified matrix, Amberlite® (iminodiacetic acid)-modified matrix, Amberlite® Type I (trialkylbenzyl ammonium)-modified matrix, Amberlite® Type II (dimethyl-2-hydroxyethylbenzyl ammonium)-modified matrix, Dowex® (polyamine)-modified matrix, Dowex Type I (trimethylbenzyl ammonium)-modified matrix, Dowex® Type II (dimethyl-2-hydroxyethylbenzyl ammonium)-modified matrix, Dowex® (mixed bed), Capto® Adhere Anion Exchange Multi Mode, PPA Hypercel, HEA Hypercel, or Duolite® (polyamine)-modified matrix.

32. The method of claim 28 wherein the mixed mode resin is Capto® Adhere Anion Exchange Multi Mode, PPA Hypercel, or HEA Hypercel.

33. A method for quantification of a polyanionic PCR inhibitor in a sample comprising:
(a) contacting a sample comprising a polyanionic PCR inhibitor with at least one aliquot of a polycationic compound;
(b) adding a polyanionic indicator dye in an amount sufficient to detect the free form of the dye; and
(c) quantifying the polyanionic PCR inhibitor based on the amount of polycationic compound needed to detect the free form of the polyanionic indicator dye.

34. A method of removing a polyanionic impurity in a sample comprising:
(a) contacting a fluid comprising a polyanionic impurity with a polycationic counterion; and
(b) separating the fluid from the polyanionic impurity complexed to the polycationic counterion, thereby removing the polyanionic impurity from the fluid.

35. The method of claim 34 wherein the polyanionic impurity is a polyanionic PCR inhibitor.

36. The method of claim 34 wherein the complex of polyanionic impurity and polycationic counterion is removed by precipitation.

37. The method of claim 34 wherein the polycationic counterion is derivatized by attachment to a member of a binding pair or a magnetic particle to facilitate removal of the complex of polyanionic impurity and polycationic counterion from the fluid.


SEQUENCE LISTING (4 sequences)

SEQ ID NO: 1 - DNA, 68 bp, Artificial Sequence
  Synthetic
gaaatcgggctgcctgagtcccgagtgcgggtgtggtttcagaaccgccgaagtcgttcggggatggt

SEQ ID NO: 2 - DNA, 19 bp, Artificial Sequence
  Synthetic
gaaatcgggctgcctgagt

SEQ ID NO: 3 - DNA, 19 bp, Artificial Sequence
  Synthetic
accatccccgaacgacttc

SEQ ID NO: 4 - DNA, 19 bp, Artificial Sequence
  Synthetic
ccgagtgcgggtgtggttt